(19) United States
(12) Patent Application Publication
(10) Pub. No.: US 2015/0292023 A1
(43) Pub. Date: Oct. 15, 2015

(54) Materials and Methods Related to NSAID Chemoprevention in Colorectal Cancer

(71) Applicants: THE OHIO STATE UNIVERSITY, Columbus, OH (US); THE GOVERNMENT OF THE U.S.A AS REPRESENTED BY THE SECRETARY OF THE DEPARTMENT OF HEALTH AND HUMAN S, Rockville, MD (US); CINCINNATI CHILDREN'S HOSPITAL MEDICAL CENTER, Cincinnati, OH (US)

(72) Inventors: Richard Fishel, Hilliard, OH (US); Carlo M. Croce, Columbus, OH (US); Levy Kopelovich, Annandale, VA (US); Bruce Aronow, Cincinnati, OH (US); Juana Martin-Lopez, Columbus, OH (US)

(73) Assignees: Ohio State Innovation Foundation, Columbus, OH (US); Cincinnati Children's Hospital Medical Center, Cincinnati, OH (US); The Government of the U.S.A. as Represented by the Secretary of the Dept. of Health & Human Services, Rockville, MD (US)

(21) Appl. No.: 14/361,940

(22) PCT Filed: Dec. 3, 2012
(86) PCT No.: PCT/US12/67651

§ 371 Date: May 30, 2014

Related U.S. Application Data
(60) Provisional application No. 61/565,540, filed on Dec. 1, 2011.

(51) Int. Cl. C12Q 1/68; G06F 19/20; A61K 31/616; G06F 19/00; A61K 31/192; A61K 45/06

(52) U.S. Cl. CPC C12Q 1/6886; A61K 31/192; A61K 45/06; A61K 31/616; G06F 19/3431; G06F 19/20; G06F 19/706; C12Q 2600/106; C12Q 2600/158

(57) ABSTRACT

Described herein are methods of preventing cancer, treating cancer, inhibiting tumor growth, predicting patient outcomes, optimizing dosage, and monitoring the efficacy of treatment. In particular, certain microRNAs and messenger RNAs are useful indicators of response to NSAID chemoprevention and therapy of patients having Lynch syndrome or hereditary non-polyposis colorectal cancer.

### CROSS-REFERENCE TO RELATED APPLICATIONS

This application claims the benefit of U.S. Provisional Patent Application No. 61/565,540, filed Dec. 1, 2011, the disclosure of which is incorporated herein by reference for all purposes.

### STATEMENT REGARDING FEDERALLY SPONSORED RESEARCH

This invention was made with U.S. Government support under Grant Number NO1-CN-43302 and NO1-CN-53301 awarded by the National Cancer Institute. The U.S. government has certain rights in the invention.

### FIELD OF THE INVENTION

This application is in the field of medicine, particularly oncology. The invention is also in the field of molecular biology, particularly microRNAs and mRNAs.

### BACKGROUND OF THE INVENTION

Lynch syndrome or hereditary non-polyposis colorectal cancer (LS/HNPCC) is the most common human cancer predisposition syndrome accounting for 3-5% of colorectal cancer (CRC) and 5-7% of endometrial and ovarian cancer. LS/HNPCC is caused by defects in the human mismatch repair (MMR) genes. In addition, 15-40% of sporadic colorectal, endometrial, ovarian, and upper urinary tract tumors display the genetic instability in simple repeat sequences (microsatellite instability or MSI) that is diagnostic of MMR defects.

Non-steroidal anti-inflammatory drugs (NSAIDs) are a structurally diverse family of compounds that can be effective in the prevention of several types of human cancer including colorectal cancer. Acetylsalicylic acid (ASA), commonly known as aspirin, is the archetype of the NSAID family. A number of epidemiological studies have demonstrated an inverse relationship between ASA use and the incidence of CRC. Recent clinical analysis showed that regular ASA use was associated with a lower risk of cancer-specific mortality in individuals already diagnosed with CRC.

Evidence suggests that long-term exposure to non-steroidal anti-inflammatory drugs (NSAIDs) reduce CRC incidence. Remarkably, a recent randomized trial with LS/HNPCC patients suggested that long-term exposure to aspirin reduced the rate of tumor incidence by at least 2-fold.

The NSAIDs, aspirin (ASA) and Sulindac, suppress MSI by a genetic mechanism that paradoxically selects for genetically stable MMR-defective cells. MSI suppression is independent of the cyclooxygenase (COX) genes 1 and 2, a common focus of CRC chemoprevention research. Nitric oxide-donating ASA (NO-ASA) suppresses MSI at doses that are 300-3000 fold lower than ASA.

Accumulating evidence indicates that NSAIDs are a potent tool against cancer. However the efficacy of any particular NSAID compound and dosage may vary considerably as compared with other NSAIDs. NSAID chemopreventive therapy may require years to indicate effective tumor inhibition based on gross examination. There is a need for additional tools and methods to measure and assess efficacy at the molecular and cellular level. Further, there is a need for tools and methods to aid in understanding the mechanism of NSAID cancer chemoprevention.

The present invention provides alternative methods of treating cancer, predicting outcomes, optimizing dosage, and monitoring the efficacy of treatment. Disclosed methods overcome limitations of conventional therapeutic methods as well as offer additional advantages that will he apparent from the detailed description below.

### SUMMARY OF THE INVENTION

Provided are methods of predicting patient outcomes, optimizing NSAID dosage, monitoring the efficacy of treatment, screening NSAID compounds for efficacy, preventing cancer, treating cancer, and inhibiting tumor growth. In particular, certain microRNAs (miRs) and messenger RNAs (mRNA) are useful indicators of response to NSAID chemoprevention and therapy of patients having Lynch syndrome or hereditary non-polyposis colorectal cancer (LS/HNPCC).

Provided are methods for characterizing a disease in a subject having, or suspected of having, LS/HNPCC comprising: comparing the level of at least one biomarker in a sample to a control, wherein the at least one biomarker is chosen from: an activation miR, an inhibition miR, an activation mRNA, an inhibition tuRNA, and combinations thereof; and, determining whether the level of at least one biomarker is reduced or whether the level of at least one miR is elevated in the sample, as compared to the control, thereby characterizing the disease in the subject.

Provided are activation miRs including: miR-136, miR-199a-3p, miR-27b, miR-377, miR-21, miR-128, miR-103, miR-34a, miR-193, miR-328, miR-296-3p; miR-19a, miR126-5p, miR-29b, miR-29a, miR-301a, miR-1, miR-218, miR142-3p, miR-190, miR-144, miR-33, miR-377, miR-1196;; mir-375-A, mir-615-P, miR-706-P, mir-485-3p-A, mir-290-P, miR-543-A, mir-425-3p-A, mir-326-A, mir-589-A, mir-129-2-A, mir-553-A, mir-96-A, mir-7-2-A, mir-519e*-5p-A, mir-293-A, has, mir-551a-A, mir-594-P, mir-601-A, miR-487b-A, miR-676-3p-A, miR-500-P;; miR-706, miR-543, miR-328, miR-202, miR-338, and miR-483.

Provided are inhibition miRs including: miR-341, miR-297c, miR-423-5p, miR762; miR-2135, miR-423-5p, miR-297c;; miR-222, miR-223, miR-130, miR-133, miR-100, miR-184, miR-135, miR-141, miR-199, miR-102, miR-143, miR-27b, miR193, and miR-9.

Provided are activation mRNAs including: RPL13A, Npas2, Arnt1, Kit1, Malat1, Ept1, 1810014B01Rik, Aes, Sepw1, Rab4a, bak1, mall, Zfand5, Nfil3, Npas2, Irf2bp21700110K17Rik; Bmpr1a, Suhw4, Msi2, Smc1a,2,5, jmjd1c, Blrc6, CdkN1b, jarid1a, and Bclaf1.

Provided are inhibition miRs including: Slc38a10, Aes, Rab5c, Cfl1, PPARD, Actb, Pttg1ip, Espn, Elovl1, Angptl2, Hlf, Per2, Tef, Per3, Dbp; Ste20a1, Gm129; Ang4; Bambi, Cd24a, Ccl6, Entpd5, Wee1, Ank3, Trp53, Sel1l, Ube4b, Arid5b, Prkab2, Per1, Bace1, and Stox2.

Provided are methods of assessing the efficacy of NSAID cancer chemoprevention treatment in a human or animal subject, the method comprising: obtaining a nucleic acid-containing sample from a subject receiving NSAID treatment; measuring miRNA expression of at least one activation miR selected from the group consisting of: miR-103, miR-193, miR-128, miR-136, miR-199a-3p, miR-27b, miR-377, miR-21, miR-34a, miR-328, miR-296-3p; determining the NSAID treatment is efficacious if the activation miR measured in the sample is activated.

Provided are methods, wherein the at least one activation miR includes: miR-103, miR-193, and miR-128.

Provided are methods of assessing the efficacy of NSAID cancer chemoprevention treatment in a human or animal subject, the method comprising: obtaining a nucleic acid-containing sample from a subject receiving NSAID treatment; measuring microRNA (miR) expression of at least one inhibition miR selected from the group consisting of: miR-341, miR-297c, miR-423-5p, miR762; miR-2135, miR-423-5p, miR-297c; determining the NSAID treatment is efficacious if the inhibition miR measured in the sample is inhibited.

Provided are methods of assessing the efficacy of NSAID cancer chemoprevention treatment in a human or animal subject, the method comprising: obtaining a nucleic acid-containing sample from a subject receiving NSAID treatment; measuring miRNA expression of at least one activation miR selected from the group consisting of: miR-19a, miR-126-5p, miR-29b, miR-301a, miR-1, miR-218, miR-142-3p, miR-190, miR-144, miR-33, miR-377, miR-1196; determining the NSAID treatment is efficacious if the activation miR measured in the sample is activated.

Provided are methods of assessing the efficacy of NSAID cancer chemoprevention treatment in a human or animal subject, the method comprising: obtaining a nucleic acid-containing sample from a subject receiving NSAID treatment; measuring miRNA expression of at least one activation miR selected from the group consisting of: miR-375-A, miR-615-P, miR-706-P, miR-485-3p-A, miR-290-Y, miR-543-A, miR-425-3p-A, miR-326-A, miR-589-A, miR-129-2-A, miR-553-A, miR-96-A, miR-7-2-A, miR-519e*-5p-A, miR-293-A, miR-551a-A, miR-594-P, miR-601-A, miR-487b-A, miR-676-3p-A, miR-500-P; determining the NSAID treatment is efficacious if the activation miR measured in the sample is activated.

Provided are methods of assessing the efficacy of NSAID cancer chemoprevention treatment in a human or animal subject, the method comprising: obtaining a nucleic acid-containing sample from a subject receiving NSAID treatment; measuring miRNA expression of at least one activation miR selected from the group consisting of: miR-103, miR-193, miR-128, miR-136, miR-199a-3p, miR-27b, miR-377, miR-21, miR-34a, miR-328, miR-296-3p, miR-19a, miR-126-5p, miR-29b, miR-301a, miR-1, miR-218, miR-142-3p, miR-190, miR-144, miR-33, miR-377, miR-1196, miR-375-A, miR-615-P, miR-706-P, miR-485-3p-A, miR-290-P, miR-543-A, miR-425-3p-A, miR-326-A, miR-589-A, miR-129-2-A, miR-553-A, miR-96-A, miR-7-2-A, miR-519e*-5p-A, miR-293-A, miR-551a-A, miR-594-P, miR-601-A, miR-487b-A, miR-676-3p-A, miR-500-P, miR-706, miR-543, miR-328, miR-202, miR-338, and miR-483; determining the NSAID treatment is efficacious if the activation miR measured in the sample is activated.

Provided are methods of assessing the efficacy of NSAID cancer chemoprevention treatment in a human or animal subject, the method comprising: obtaining a nucleic acid-containing sample from a subject receiving NSAID treatment; measuring messenger RNA (mRNA) expression of at least one activation mRNA selected from the group consisting of: RPL13A, Npas2, Arnt1, Kit1, Malat1, Ept1, 1810014B01Rik, Aes, Sepw1, Rab4a, bak1, mall, Zfand5, Nfil3, Npas2, and Irf2bp21700110K17Rik; determining the NSAID treatment is efficacious if the activation mRNA measured in the sample is activated.

Provided are methods wherein the at least one activation mRNA preferentially includes Kit1.

Provided are methods of assessing the efficacy of NSAID cancer chemoprevention treatment in a human or animal subject, the method comprising: obtaining a nucleic acid-containing sample from a subject receiving NSAID treatment; measuring messenger RNA (mRNA) expression of at least one inhibition mRNA selected from the group consisting of: Slc38a10, Aes, Rab5c, Cfl1, PPARD, Actb, Pttg1ip, Espn, Elovl1, Angptl2, Hlf, Per2, Tef, Per3, Dbp; Ste20a1, and Gm129; determining the NSAID treatment is efficacious if the inhibition mRNA measured in the sample is inhibited.

Provided are methods further comprising measuring at least one inhibition mRNA selected from the group consisting of: Ang4; Bambi, Cd24a, Cc16, Entpd5, Wee1, Ank3, Trp53, Sel1l, Ube4b, Arid5b, Prkab2, Per1, Bace1, and Stox2.

Provided are methods of assessing the efficacy of NSAID cancer chemoprevention treatment in a human or animal subject, the method comprising: obtaining a nucleic acid-containing sample from a subject receiving NSAID treatment; measuring at least one mRNA and/or miR biomarker; determining whether the NSAID treatment is efficacious based on the expression of the at least one mRNA and/or miR biomarker.

Provided are methods wherein the sample is extracted from at least one of the group consisting of: tissue, blood, tumor, stool, mucus, dysplastic tissue, colorectal tissue, ovarian tissue, urothelial tissue, endometrial tissue, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof.

Provided are methods wherein the measuring is performed by one or more methods selected from the group consisting of: hybridization assay, microarray chip, n-counter (NanoString) analysis, miR-Seq analysis, PCR, real-time PCR, microfluidic cards, oligonucleotide probes, and northern blot.

Provided are methods wherein the determining further comprises comparing the sample expression to a control level, wherein the control level is determined from measurements of control expression levels in tissue selected from the group consisting of: cancerous tissue, healthy tissue, and tissue taken from the subject at an earlier time, wherein the earlier time is greater than any of: one week, one month, six months, 200 days, one year, 400 days, eighteen months, 600 days, two years, three years, four years, five years, six years, seven years, eight years, nine years, ten years, and eleven years.

Provided are methods of inhibiting tumors that display microsatellite instability (MSI) in a human or animal subject comprising administering an efficacious NSAID for a period exceeding five years.

Provided are methods wherein the NSAID is Naproxen.

Provided are methods of inhibiting tumor incidence in hereditary non-polyposis colorectal cancer or Lynch syndrome comprising: administering a therapeutically effective dose of non-steroidal anti-inflammatory drug (NSAID); and monitoring the efficacy of the treatment by measuring at least one biomarker selected from the group consisting of: an activation miR, an inhibition miR, an activation mRNA, and an inhibition mRNA.

Provided are methods of inhibiting tumor progression in Lynch syndrome or hereditary non-polyposis colorectal cancer (LS/HNPCC) comprising: administering a therapeutically effective dose of non-steroidal anti-inflammatory drug (NSAID); and monitoring the efficacy of the treatment by measuring at least one biomarker selected from the group consisting of: an activation miR, an inhibition miR, an activation mRNA, and an inhibition mRNA.

Provided are methods of treating LS/HNPCC comprising: administering a therapeutically effective dose of non-steroidal anti-inflammatory drug (NSAID); and monitoring the efficacy of the treatment by measuring at least one biomarker selected from the group consisting of: an activation miR, an inhibition miR, an activation mRNA, and an inhibition mRNA.

Provided are methods wherein the NSAID is naproxen and/or naproxen sodium.

Provided are methods wherein the NSAID is selected from the group consisting of: naproxen, naproxen sodium, nitric oxide-donating acetylsalicylic acid, sulindac, nitric oxide-donating sulindac, fenoprofen, ketoprofen, oxaprozin, indomethacin, etodolac, diclofenac, piroxicam, meloxicam, tenoxicam, droxicam, lomoxicam, isoxicam, mefenamic acid, meclofenamic acid, flufenamic acid, tolfenamic acid, celecoxicab, rofecoxib, valdecoxib, parecoxib, lumiracoxib, etoricoxib, nimesulide, and niflumic acid, and licofenac.

Provided are methods wherein at least two NSAIDs are administered.

Provided are methods of determining NSAID dosage for treating LS/IINPCC comprising: administering a dose of non-steroidal anti-inflammatory drug (NSAID); monitoring the efficacy of the treatment by measuring at least one biomarker selected from the group consisting of: an activation miR, an inhibition miR, an activation mRNA, and an inhibition mRNA; and thereafter increasing or decreasing the dose to therapeutically effective levels, whereby the therapeutically effective level is determined by comparing the at least one biomarker with a control.

Provided are methods for screening a NSAID compound comprising: administering a test NSAID compound to an animal subject; measuring at least one biomarker selected from the group consisting of: an activation miR, an inhibition miR, an activation mRNA, and an inhibition mRNA; and comparing the test compound biomarker expression profile with a biomarker expression profile for a NSAID compound of tested efficacy.

Provided are methods for the assessment of a clinical condition related to cancer of a patient for in vitro diagnosis of cancer, comprising: obtaining a sample from a subject; determining expression levels of one or more miRNAs as cancer biomarkers and an internal control RNA; computing relative expression levels of the one or more miRNAs as cancer biomarkers; computing a prediction model by using one or more variables, wherein the variables include relative expression levels of the one or more miRNAs as cancer biomarkers; and, computing a cancer progression risk probability by the prediction model, wherein the subject is diagnosed as at risk for cancer progression if the disease risk probability is greater than 0.5.

Provided are methods for providing a prognosis for survival in a subject having LS/HNPCC, comprising: comparing the level of at least biomarker level in a sample to a control, wherein the at least one biomarker is chosen from: an activation miR, an inhibition miR, an activation mRNA, and an inhibition mRNA, and combinations thereof; and determining whether the level of at least biomarker is reduced or whether the level of at least one biomarker is altered in the sample, as compared to the control, thereby characterizing a prognosis for survival in a subject having LS/HNPCC.

Provided are methods to determine progression of LS/HNPCC in a subject, comprising: comparing the level of at least one biomarker in a sample to a control, wherein the at least one biomarker is chosen from: an activation miR, an inhibition miR, an activation mRNA, an inhibition mRNA, and combinations thereof; and determining whether the level of at least one biomarker is reduced or whether the level of at least one biomarker is elevated in the sample, as compared to the control, thereby determining progression of LS/HNPCC in the subject.

Provided are diagnostic kits of molecular markers for providing a prognosis for poor survival of a subject having LS/HNPCC, according to any of the above methods, the kit comprising a plurality of nucleic acid molecules, each nucleic acid molecule encoding a microRNA and/or messenger RNA sequence, wherein at least one miRNA sequence is miR-103; and wherein one or more of the plurality of nucleic acid molecules are differentially expressed in the target cells and in one or more control cells, and wherein the one or more differentially expressed nucleic acid molecules together represent a nucleic acid expression signature that is indicative of prognosis of poor survival in a subject having LS/HNPCC.

Various objects and advantages of this invention will become apparent to those skilled in the art from the following detailed description of the preferred embodiment, when read in light of the accompanying drawings.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Randomized Trial of Aspirin Chemoprevention in LS/HNPCC Patients. Data from the Colorectal Adenoma-Carcinoma Prevention Program (CAPP). LS/HNPCC patients were randomized and provided with either a placebo or 600 mg aspirin daily. Tumor location and number were determined by colonoscopy on a yearly basis. Hazard Ratio (HR)=0.61(0.1 9, 0.86) and p=0.02. Aspirin, 600mg/day shown on the bottom red line; placebo shown by the blue line above.

FIGS. 2A-B. NSAID Treatment Increases Survival of LS/HNPCC Mice. Msh2^{flox/flox},VpC^{+/+} (LS/HNPCC) and VpC⁺⁺ (wild type) were treated with eight NSAIDs at two doses. Representative survival data is shown and separated into (FIG. 2A) No Effect (Sulindac Sulfone and NO-Sulindac Sulphone), (FIG. 2B) Modest Effect (20-50% increase in life span; Aspirin, Sulindac, NO-Sulindac), and (FIG. 2C) Naproxen (˜100% increase in life span). The survival of untreated Msh2^{flox/flox}VpC^{+/+} animals is shown in squares (red). Aspirin treatment is shown with black dots for comparison. Treatment of selected wild type controls is shown with triangles (purple). Note: While Sulindac treatment increases lifespan similar to aspirin, it also induces tumors in the wild type controls (FIG. 2B).

FIGS. 3A-3E. Tumor Size, Number and Pathology in LS/HNPCC mice treated with Aspirin. (FIG. 3A) Representative gross intestinal lesion from treated mice. (FIGS. 3B-E) Representative examples of tumors from Msh2^{flox/flox}, VpC^{+/+} mice showing aberrant crypt foci, gastrointestinal intraepithelial neoplasia and adenocarcinoma. In several cases we have noted tumor infiltrating leukocytes; a common pathological observation in LS/HNPCC tumors.

FIGS. 4A-4B. Comparative MSI summary of intestinal tumors from Msh2^{flox/flox}VpC^{+/+} mice following ASA treatment. Ear (E), intestinal tumor (T), and adjacent normal intestinal tissues to the tumor (N) were examined for microsatellite changes. Average allele distributions are shown in colors. (FIG. 4A) Untreated LS/HNPCC mice. (FIG. 4B) Aspirin treated LS/HNPCC mice. As expected, the E tissues are microsatellite stable (MSS) under all conditions since the Msh2 knockout is confined to the intestinal tissues. Untreated mice display MSI in the intestinal tissues that are increased in the tumor (FIG. 5B), while aspirin treated mice display decrease MSI in both the tumor and adjacent normal tissues.

FIG. 5. Heat map showing 115 Sa miR microarray with Naproxen mutants affect and differential versus sulindac sulfone-NOsulindac sulfone. Yellow shows increased expression; blue shows reduced expression.

FIG. 6. Heat map showing 115 Sa miR microarray with Naproxen mutants affect and differential versus sulindac sulfone-NOsulindac sulfone BIG.

FIG. 7. Heat map showing 61 miR Affy signature naprox ves sulfone ave Heat map.

FIG. 8. Heat map showing 29 miRGE 84 nanostring survey MSH mutant therapeutic drug effects.

FIG. 9. Heat map showing 15 miR nanostring 24 big library survey study Naproxen On1V3.

### DETAILED DESCRIPTION

Lynch syndrome or hereditary non-polyposis colorectal cancer (LS/HNPCC) is the most common cancer predisposition syndrome in the human population and is found in nearly 1 in 300 individuals. LS/HNPCC is caused by alterations of the mismatch repair (MMR) genes. Tumors evolve from the increased mutations normally suppressed by MMR. A large fraction of spontaneous colorectal (15%), endometrial (30%), and upper urinary tract (40%) tumors also display length changes in normally stable short DNA sequence repeats (microsatellite instability or MSI) that is a diagnostic indicator of MMR defects. Greater than 90% of LS/HNPCC is caused by unambiguous mutations in human MMR genes.

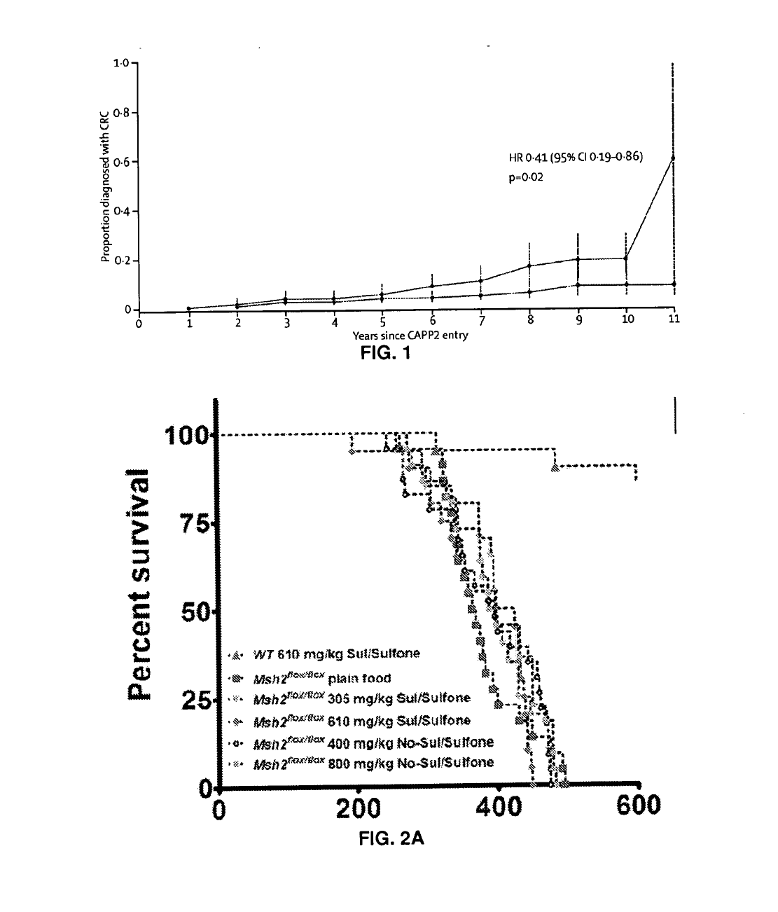

The Colorectal Adenoma-Carcinoma Prevention Program (CAPP) has examined the potential of aspirin (acetylsalicylic acid: ASA) to reduce colorectal neoplasia in LS/HNPCC carriers in a randomized trial (FIG. 1). The end-point for these studies was the detection of LS/HNPCC cancers during yearly colonoscopy screens. Remarkably, the benefits for LS/HN PCC patients begins after 5 yrs of ASA exposure and results in at least a 2-fold decrease in the rate of tumor formation compared to the placebo-treated cohort.

Inhibition of cyclooxygenase-1 (COX1) or COX2 activity that is involved in the production of inflammatory prostaglandins was, in the past, generally considered a mechanism for cancer chemoprevention by NSAIDs. However, it was later demonstrated that long-term exposure of MMR-deficient human colon tumor cells to ASA suppressed MSI via a genetic selection that enhance apoptosis in critically unstable cells. Ultimately, ASA treatment resulted in a cell population that had a persistent MMR deficiency, but had paradoxically acquired a largely microsatellite stable (MSS) phenotype. The selection for MSS cells was independent of the COX1 or COX2 genes.

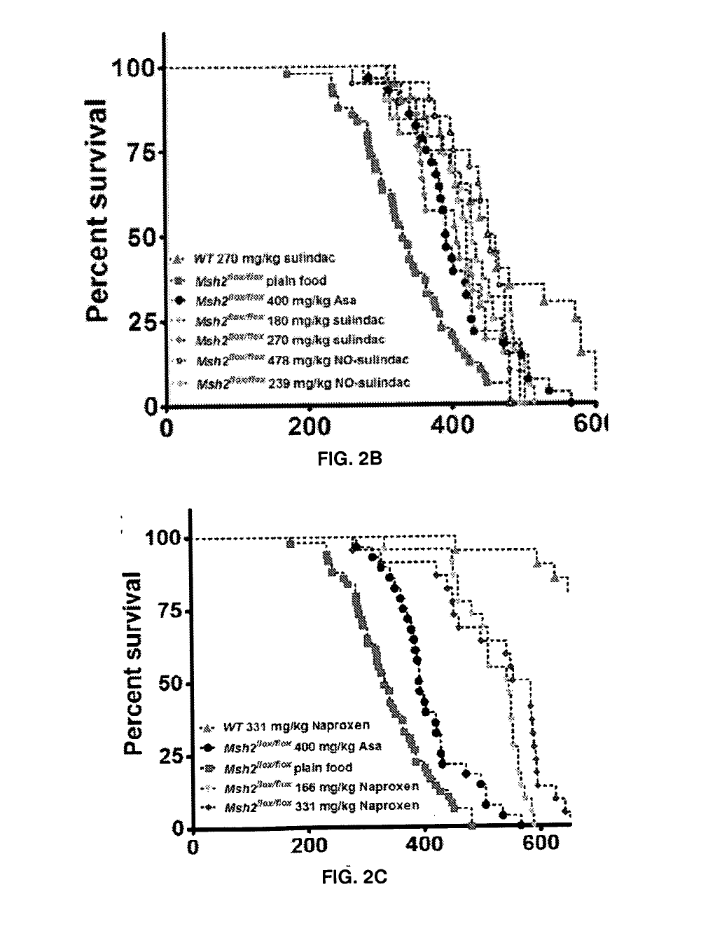

Treatment of MMR-defective cells with (ASA or sulindac suppressed MSI by the proapoptotic genetic selection mechanism. These results show that ASA and sulindac is useful cancer chemopreventives for LS/HNPCC carriers. In addition, ASA containing a nitric oxide (NO) donating group suppressed MSI cells at doses 300-3000 fold lower than ASA (FIG. 2). Using a mouse model for LS/HNPCC intestinal cancer, results showed that ASA and low dose NO-ASA suppressed tumorigenesis and increased survival.

Described herein are the effect of eight different NSAIDs at multiple doses in LS/HNPCC cellular and mouse model systems. A wide-range of chemoprevention efficacy is shown, including results ranging from no effect to substantial effects. Remarkably, treatment of the LS/HNPCC mouse model with naproxen (also known as naprosyn) substantially increases survival such that these animals live nearly double the life span of untreated animals. The naproxen-treated LS/HNPCC mice live a nearly normal life. This represents a notably large effect of a chemopreventive on survival for a mouse cancer model.

The mouse model for LS/HNPCC shows analogous intestinal tumor pathology to human LS/HNPCC. Likewise, long-term administration of ASA and NO-ASA to LS/HNPCC mouse models increased survival similarly to human patients. A substantial NSAID chemoprevention effect on a common genetic cause of cancer that is recapitulated from the mouse model to the human disease provides a unique tool capable of dissecting the molecular mechanisms responsible for NSAIDs cancer chemoprevention efficacy.

Because there is such wide-range of NSAID effects, it is important to develop mRNA and miRNA expression signatures that correlate with efficacious NSAID-induced survival. This expression signature may be extracted from multiple independent overlapping and confounding NSAID cellular effects.

Experimental Methods

Treatment with Some NSAIDs Increased Survival of LS/HNPCC Mice

Cohorts of equivalent gender Msh2^{flox/flox}VpC^{+/+} mice were treated with NSAIDs including ASA that were mixed with powdered diet and administered ad libitum. VpC^{+/+} (Msh2⁺⁺) mice were similarly examined to control for potentially confounding effects of continuous Cre expression in the intestine. Kaplan-Meier survival analyses were generated for each NSAID treatment (FIG. 2). The median survival time of the untreated Msh2^{flox/flox}VpC^{+/+} mice was 333 days (red squares; FIG. 2). In contrast, treatment with 400 mg/kg ASA increased the mean survival of the LS/HNPCC mice to 393 days, equivalent to an 18% increase in lifespan (black dots, p<0.001; FIG. 2B and FIG. 2C). These results display a similar tumor suppression trend to that observed in a randomized placebo controlled study of the effect of aspirin on heterozygous human LS/HNPCC carriers. Cohorts of the LS/HNPCC mice were treated with seven additional NSAIDs and NO-NSAID derivatives, many of which have reported chemopreventive activity in other mouse models as well as humans. NSAID treatment incorporated at least two doses below the minimum tolerated dose (MTD) for mice and included: NO-ASA, Naproxen, NO-Naproxen, sulindac, NO-sulindac, sulindac sulfone, and NO-sulindac sulphone. Representative survival data is separated into little or no-effect (FIG. 2A), modest effect (20-50% increased life span; FIG. 2B), and significant effect (>70% increase in lifespan; FIG. 2C) on survival. The mean survival of LS/HNPCC mice treated with 72 (mg/kg NO-ASA was 403 days; a comparable survival to ASA but at a 10-fold lower dose. Sulindac and NO-sulindac also increased survival similar to ASA (FIG. 2B). However, both of these compounds significant reduce the survival of the wild type mice (triangles, FIG. 2B). Interestingly, the results have not shown more than a —20% increase in survival over a wide range of ASA and NO-ASA doses. In contrast, the mean survival of LS/HNPCC mice treated with naproxen was 545 days and 569 days for 166 mg/kg and 331 mg/kg respectively. The lack of a substantial difference between the two doses of naproxen treatment suggests that lower doses are likely to provide protection.

Tumor Pathology Following NSAID Treatment

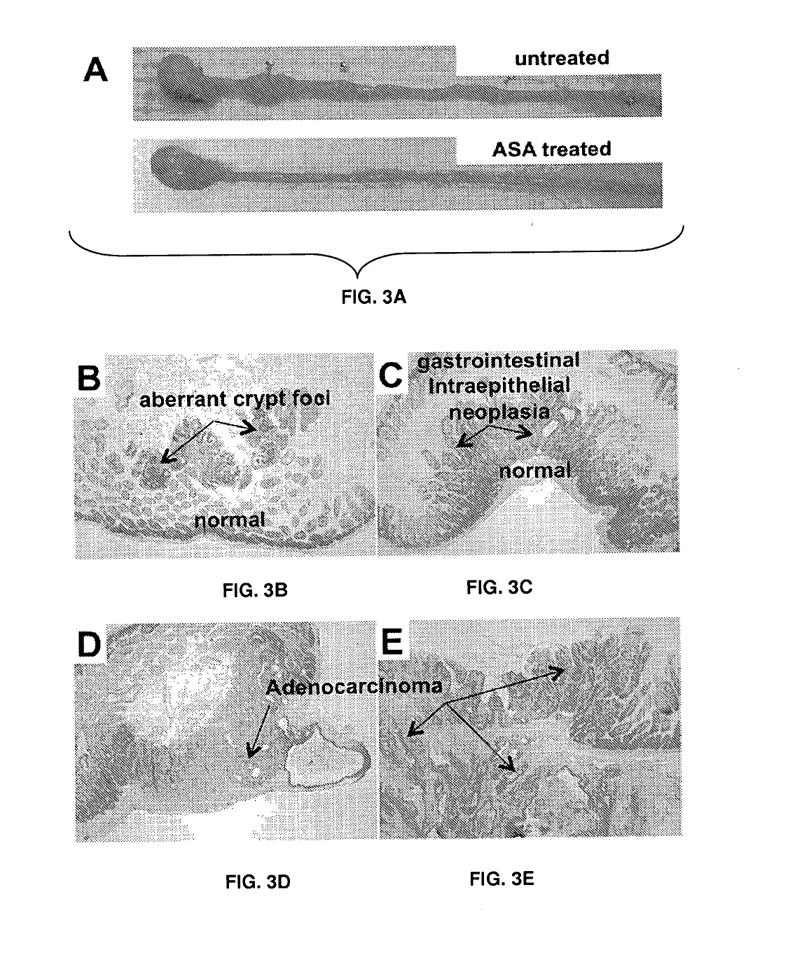

Tumors are confined to the intestines of the LS/HNPCC mice, predominantly in the duodenum and jejunum, and rarely in the ileum; consistent with the pattern and levels of villin expression in these tissues (FIG. 3A). Remarkably, sulindac treatment increases the number of cecal cancers as has been reported for other mouse models. Although mice treated with ASA, NO-ASA and naproxen presented with morbid tumors significantly later than untreated mice, when sacrificed they had statistically equivalent numbers of tumors compared to the untreated mice (1.68±0.72, 1.78±1.25, and 1.58±0.67 versus 1.90±1.02, respectively). Intestinal lesions and tumors included aberrant crypt foci, adenomas and adenocarcinomas similar to human pathology (FIGS. 3B-3E). Taken together, these results indicate that efficacious NSAIDs acts as a chemopreventive by delaying tumorigenesis, but the tumors that ultimately arise appear to display similar gross pathology to untreated controls.

Examination of Tissues

Mouse tissues requiring pathologic or morphometric analysis are prepared and analyzed. For intestinal pathology, mice are euthanized and the intestinal tract isolated. The entire length of the intestine is opened and examined for visible lesions. The location, size and number of tumors are recorded. Gross analysis includes examination and characterization of all other organs for metastasis or primary lesions. Organs are then fixed in 10% neutral buffered formalin, dehydrated through a gradient of alcohols, and embedded in paraffin. Tissues will be processed using routine techniques in histology or adaptations thereof. For routine screening of the mice, histologic analysis is performed on 38 tissue sections representing all major organ systems. Sections are cut at 4-5μ and stained with hematoxylin and eosin. For analysis of proteins/enzymes, which are destroyed or degraded by routine fixatives, frozen sections are examined. Frozen sections are embedded in OCT and flash frozen in liquid nitrogen. Samples are stored at −70° C. until sectioning on a Jung cryostat. When tissue specific analysis is indicated, only tissues from the specified organs are collected and analyzed. Of particular importance is the analysis of the intestinal tract. Sequential longitudinal sections of the mouse intestine are typically examined so as to incorporate the entire intestine with orientation maintained. This technique effectively maintains the proximal-distal orientation without excessive cutting of the tissues on 4 slides.

Critical to analysis of tissue samples in all projects is the accurate identification and characterization of gastrointestinal neoplasms. Standard diagnostic criteria are used for identification of pre-neoplastic and neoplastic lesions based on previously established lesion characteristics in mice and rats. Since spontaneous neoplasms of mice differ between strains, the use of control littermates for these studies is critical.

Gross examination of the mouse may reveal distinct lesions in multiple organ systems. Of specific interest are metastatic lesions to the mesentery (including mesenteric lymph nodes), liver, and lungs. Any visible lesions are measured and enumerated. These same organs are examined histologically on the mice to confirm the absence of microscopic metastasis. Whole body analysis of representative animals can also be performed to confirm the absence of metastasis to less common locations, for example to the heart, pancreas, spleen, peripheral lymph nodes or kidneys. The whole body analysis is used to evaluate other disease development in these unique animal models.

Morphometric Analysis

Morphometric analysis can be used to discriminate differences in tumor size, or tumor frequency. Slides arc examined on an Olympus BX60 microscope with lens magnification of 1-100× dry. Using an Olympus DP72 camera images are captured and using morphometric or stereologic techniques to identify individual cells or lesions in a field, which are positive for a colorimetric change. Fully automated counting techniques or “human” assisted counting may be used. With either collection technique, an unbiased sampling of the population is obtained by using strict adherence to standard protocols for grid selection, and counting techniques. The pattern is described as a systematic random sampling technique.

Referring now to the following Figures for the following discussion:

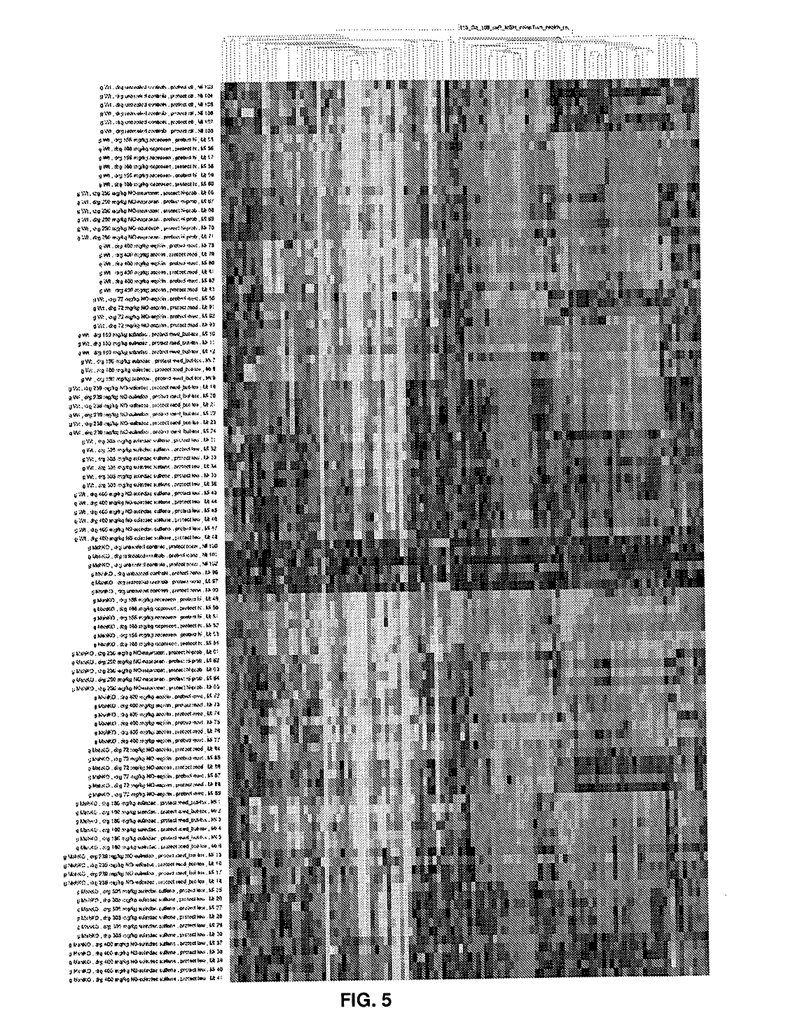

FIG. 5 shows a heat map showing 115 Sa miR microarray with Naproxen mutants affect and differential versus sulindac sulfone-NOsulindac sulfone.

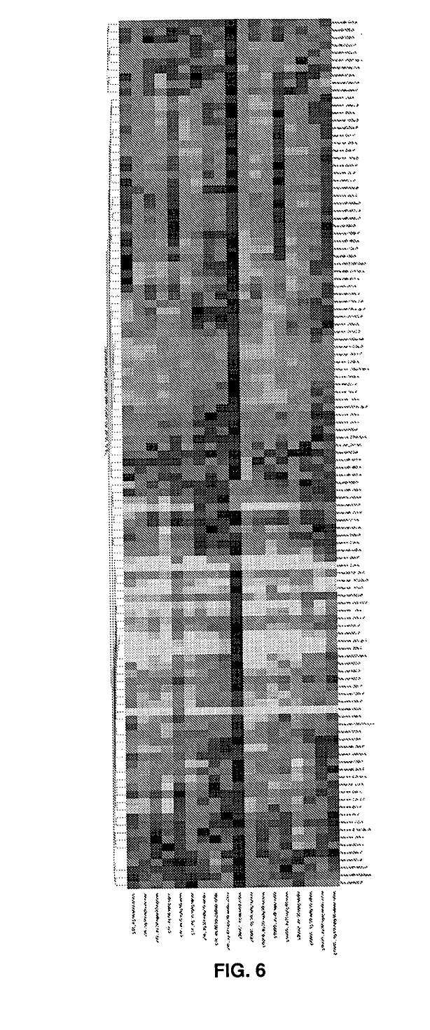

FIG. 6 shows a heat map showing 115 Sa miR microarray with Naproxen mutants affect and differential versus sulindac sulfone-NOsulindac sulfone BIG.

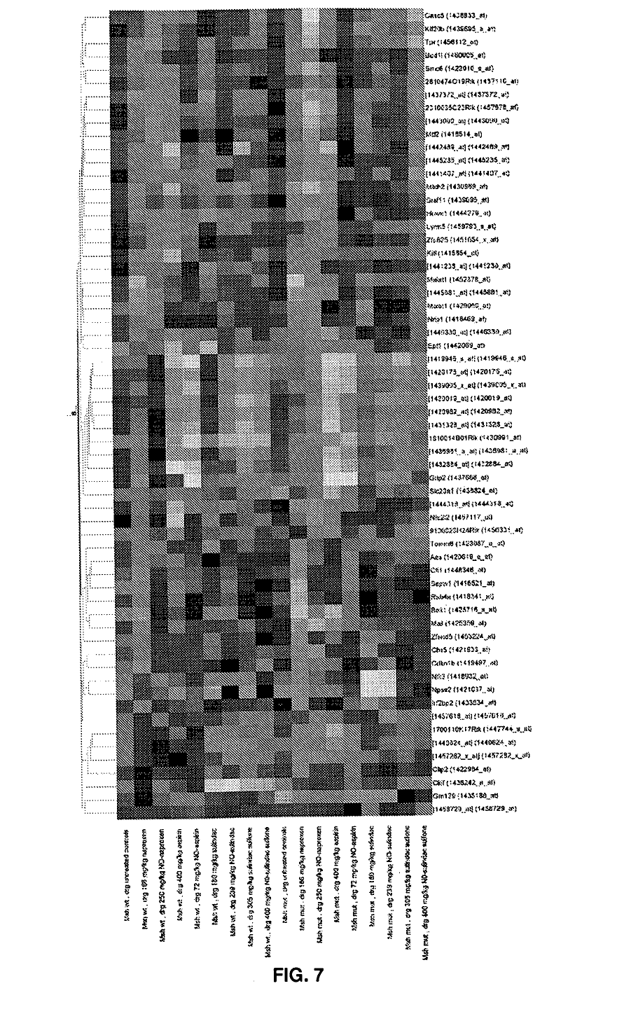

FIG. 7 shows a seat map showing 61 miR Affy signature naprox ves sulfone ave Heat map.

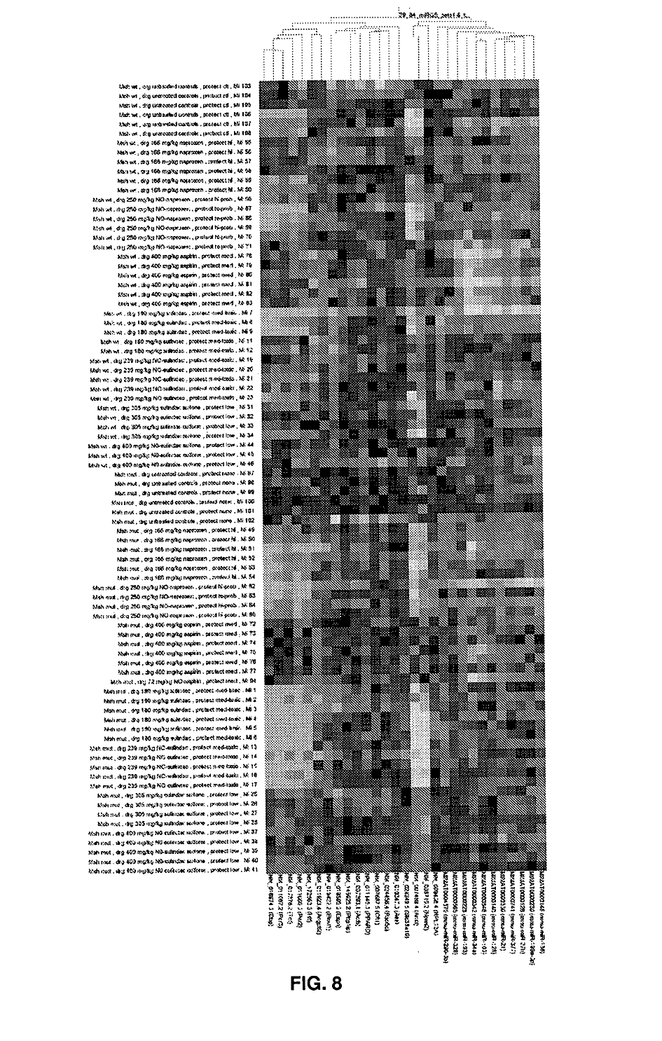

FIG. 8 shows a heat map showing 29 miRGE 84 nanostring survey MSH mutant therapeutic drug effects.

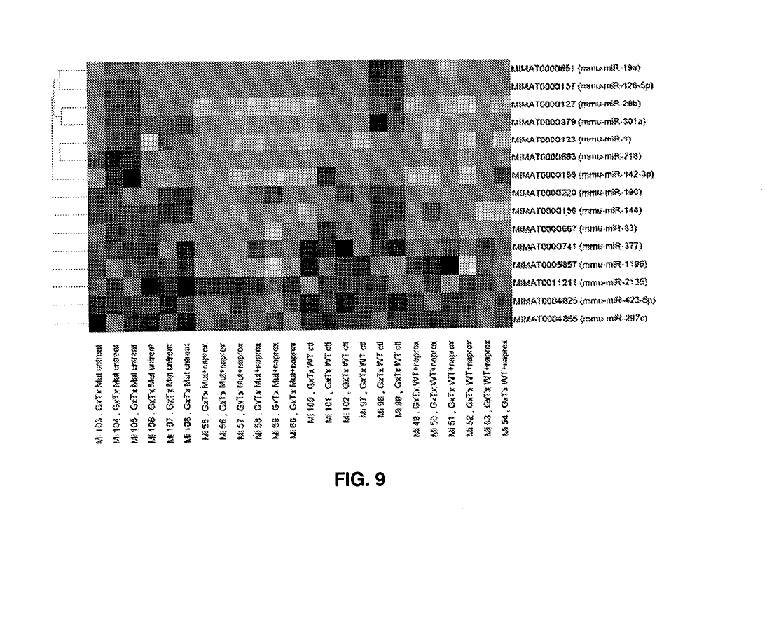

FIG. 9 shows a heat map showing 15 miR nanostring 24 big library survey study Naproxen On1V3.

### Determining the Mechanism of NSAID Suppression of LS/HNPCC Tumors

The profiled differential rates of tumor development across a large cohort of animals are analyzed to define mRNA and miRNA expression changes associated with NSAIDs that increase lifespan and NSAIDs that do not. Pathway biomarkers associated with NSAID tumor suppression are validated by real-time PCR and nCounter digital analysis. Selected pathway biomarkers amenable to validation with immunohistochemical probes are further useful in pathology analysis. Detaining relative effects is accomplished by examining the dose- and time-dependent changes in intestinal mRNA and miRNA following treatment with aspirin, naproxen, and other NSAIDs to detail the relative effects as well as refine and expand the panel of NSAID biomarkers. These studies are correlated with survival to further refine the panel of pathway biomarkers. Further to this analysis is to construct tetₒₙ-tet_{off} expression system for selected pathway biomarkers to determine their effect on MSI in MMR-defective cells. These studies identify the molecular pathways, their interactions, and mechanisms that contribute to NSAID tumor suppression.

NSAID Treatment Reduces Microsatellite Instability (MSI) in Tumor Tissues

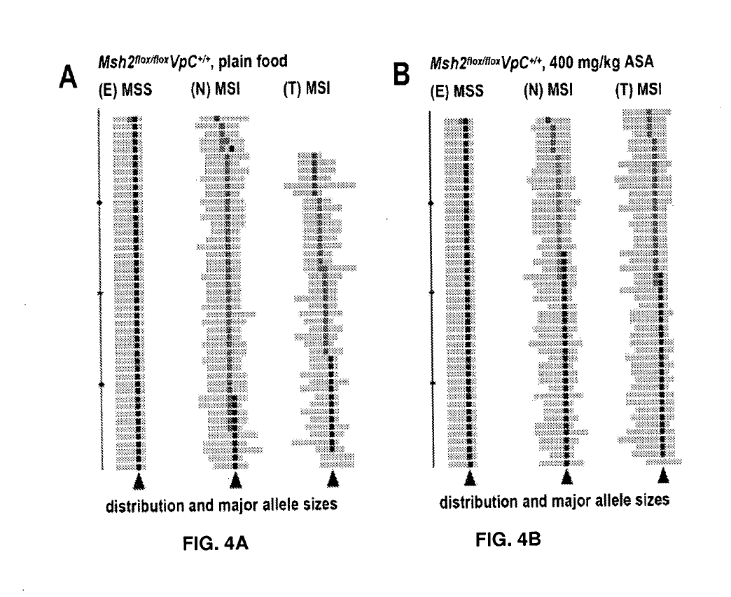

A MSI analysis on tissues from the ASA and NO-ASA treatment groups by small pool PCR (1-10 genomic equivalents) was performed to determine the distribution of novel alleles between constitutional DNA (Ear, E) as well as normal (N) and tumor (T) intestinal tissues. When multiple samples of each tissue were analyzed, the resulting profiles comprised a representational distribution of the intrinsic allelic diversity. Ear tissues from these mice were consistently MSS, with the major allele always flanked by a relatively unvarying pattern of alleles that arise from PCR amplification-artifacts. Normal and intestinal tissues from ASA and NO-ASA treated mice show variation in major allele sizes and distributions. However, it is clear that treatment with ASA and NO-ASA at least partially converted MSI to MSS in the intestinal compartment (compare FIG. 4A with FIG. 4B).

A comparative assessment of MSI status was made between intestinal tumors and their matched adjacent normal tissues by determining the absolute changes in microsatellite alterations between them. Comparative MSI provides a quantitative measure of the relative differences in instability between adjacent developmentally related normal and tumor tissues. The Bethesda Criteria for diagnostic microsatellite instability was used: MSS, no changes in microsatellite marker status; high microsatellite instability (MSI-H; ≧2 of 5 markers different). Untreated animals had a comparative MSI-H proportion of 33% whereas ASA treated LS/HNPCC mice displayed an MSI-H of 22% and NO-ASA treated mice displayed an MSH-H of 20%. This analysis suggested treatment with ASA and NO-ASA partially stabilize MSI in the intestinal epithelia similar to the cellular studies.

Analysis of mRNA and miRNA Following NSAID Treatment

Groups of 12 mice were continuously treated (6 VpC^{+/+} wild type and 6 Msh2^{flox/flox}VpC^{+/+} LS/HNPCC mice) with each of the eight NSAIDs and the intestines were harvested after two months exposure. A 0.5 cm section of intestine 2.5 cm proximal of the cecum was harvested from each individually treated mouse. High quality mRNA and miRNA was prepared for expression array analysis. To reduce the possibility of day-to-day variation the hybridization of all samples was performed simultaneously as well as sequential expression analysis. Transcriptome expression data from both the mRNA and miRNA chips was analyzed. Analysis of the data suggests superb consistency between replicate samples.. These observations underline the high quality of the mRNA and miRNA preparation methods as well as chip analysis.

Software development contributes to the data analysis. Most programming is done using Java using Eclipse and using the Struts framework with statistical and mathematical functions carried out using the R-Server application programming interface. Jakarta Project's Struts Framework allows for the use of Servlets, JSP, custom Struts tag libraries and other components using a unified framework that helps rapid design and deployment of web applications. The value of frameworks is realized in the ordered and componentized nature of code that facilitates maintenance, update, and reuse and Tomcat deployments. Advanced programming developer tools (Eclipse, IDE, CVS, Ant, compilers, debuggers, graphical developments environments) and parallel computing software (MPI, PVM) are also used extensively.

Interestingly, both Msh2 status, and treatment with NSAIDs have strong impacts on the mRNA and miRNA expression profiles. Using Benjamini-Hochberg false discovery corrected p-values in a mixed effect ANOVA model, 297 Affymetrix MOE430 plus two probe sets corresponding to 197 independent genes and 28 miRNA probes on the Ohio State miRNA oligo platform, exhibited expression changes in the LS/HNPCC mouse intestinal tissue. Differentially regulated mRNAs and miRNAs that displayed statistically significant effects as a result of efficacious NSAID treatment were then subjected to cluster analysis.

Two major patterns emerged. First, the majority of mRNAs and miRNAs expression changes induced by NSAIDs that are efficacious for tumor chemoprevention showed little or no change in response to NSAIDs that are ineffective at reducing tumorigenesis. This observation is an important step in identifying candidate chemoprevention biomarkers that have clinical utility. Second, many of these mRNAs and miRNAs were differentially expressed in the LS/HNPCC mouse intestinal tissues (Msh2^{flox/flox}VpC^{+/+}) compared to the wild type intestinal tissues (VpC^{+/+}). These results indicate a number of adaptive expression changes that are associated with deletion of the Msh2 alleles. Such expression changes were unexpected since the MSH2 protein is only known to function in MMR and DNA damage sensing. Several of the mRNAs that were induced by deletion of Msh2 are members of potential compensatory DNA repair pathways that include several damage response genes including ATM, appear to be enhanced.

A panel of mRNA and miRNA that provide a signature for NSAID chemoprevention in the LS/HNPCC mouse were identified . Remarkably, many of the chemopreventive NSAID-induced mRNA and miRNA expression changes of this panel revert to the expression pattern found in the wild type cells (compare WT an MUT expression pattern with “Effective” NSAID expression pattern. The miRNA expression signature appears most indicative of NSAID-induced expression reversion . Without wishing to be bound by theory, these results are consistent with the hypothesis that efficacious NSAID chemoprevention provokes a readaptation of cellular expression to a normal pattern. Conversely, mutations of tumor suppressors including Msh2 incite wide-ranging and perhaps compensating cellular expression changes that would not be considered a part of their specific cellular functions. Moreover, some of these compensating expression changes may help to drive tumorigenesis and may be reversed by efficacious NSAIDs. Extrapolating from this theory, several important predictions about the role of NSAIDs in cancer chemoprevention can be made. First and foremost is that these reversible expression changes are NSAID dose-dependent and the dose dependence correlates with the chemopreventive effect. Second, several of these expression changes alter tumor pathways that may evolve during cancer development and perhaps the molecular pathology of the developing tumor.

Since many of the mRNA expression changes demonstrate a reversion from LS/HNPCC (MUT) patterns back to the pattern exhibited by the wild type (WT) mouse tissues, the focus was on candidates with the largest expression changes. These represent mRNAs that are elevated or decreased by efficacious NSAID exposure. Bmpr1a, bone morphogenetic protein type I receptor, belongs to a class of receptor serine/threonine kinases that bind members of the TGFβ superfamily of ligands, which regulate cell growth. The TGFβORII receptor is mutated in more than 80% of LS/HNPCC tumors. KitL, KIT ligand, is a cytokine that binds to the c-Kit receptor. It plays an important role in hematopoiesis, spermatogenesis, and melanogenesis. Activating mutations of c-Kit are associated with gastrointestinal stromal tumors.

Identifying functional and regulatory connectivity among the differentially regulated miRs and mRNAs was analyzed to begin to determine biological pathways and networks responsible for NSAID-mediated tumor chemoprevention,. A combination of tools were used to deconvolute network interactions, including the ToppGene, ToppCluster, and ToppMir tools. The results of these analyses indicate a tremendous degree of connectivity among the differentially regulated mRNAs and miRNAs. Moreover, there is a strong relationship between the dysregulated mRNAs of genes associated with colorectal cancer risk to the differentially regulated miRNAs that target those differentially regulated mRNAs (see light boxes at top of network diagram). For example, the miR-9 and miR-135a target SMC1a. Both of these miRNAs are down-regulated by naproxen, while the SMC1a mRNA expression is enhanced by naproxen. Also noted are multiple points of connectivity with cMyc and MYC/MAX expression pathways that are commonly altered during colorectal tumorigenesis (see light boxes at bottom of network diagram). These observations suggest overlapping network mechanisms for regulating the expression of several genes.

General Methods

Microarray is a powerful high throughput tool capable of monitoring the expression of thousands of m RNAs or miRNAs at once across tens of samples processed in parallel in a single experiment. Resources utilized include the Nucleic Acids Shared Resource (NASR) and the MicroArray Shared Resource (MASR) that is part of the Ohio State University (OSU) Comprehensive Cancer Center (CCC), to conduct genome-wide analysis of mRNA and miRNA expression of treated and untreated samples. NASR and MASR are useful in variety of microarray applications for cancer research including genome-wide mRNA expression analysis on Affymetrix GeneChips, as well as genome-wide/targeted mRNA and miRNA expression analyses on custom arrays as well as Applied Biosystems Microfluidic Cards.

The miRNA microarray platform developed at OSU has enabled the MASR to profile more than fifteen thousand of cancer samples. Verification of mRNA and miRNA expression patterns is performed using real-time PCR and ultimately the nCounter digital analysis system (NanoString Technologies). The nCounter Analysis System utilizes a novel digital technology that is based on direct multiplexed measurement of gene expression and offers high levels of precision and sensitivity (<1 copy per cell). The technology uses molecular “barcodes” and single molecule imaging to detect and count hundreds of unique transcripts in a single reaction.

mRNA expression profiling—Total RNA isolation uses the TRIzol method (Invitrogen), according to the manufacturer's instructions. GeneChip Mouse genome 430 2.0 arrays (Affymetrix); containing probe sets for >45,000 characterized genes and expressed sequence tags is useful. Sample labeling and processing, GeneChip hybridization, and scanning have been performed according to Affymetrix protocols. Studies were completed to reduce the mRNA isolation from tissues to practice, and to determine the minimal number of samples required for statistical power. Briefly, double-stranded cDNA was synthesized from total RNA with the SuperScript Choice System (Invitrogen), with a T7 RNA polymerase promoter site added to its 3′ end (Genset, La Jolla, Calif.). Biotinylated cRNAs were generated from cDNAs in vitro and amplified by using the BioArray T7 RNA polymerase labeling kit (Enzo Diagnostics). After purification of cRNAs by the RNeasy mini kit (Qiagen, Hilden, Germany), 20 μg of cRNA was fragmented at 94° C. for 35 min. Approximately 12.5 tg of fragmented cRNA was used in a 250-μl hybridization mixture containing herring-sperm DNA (0. 1 mg/ml; Promega), plus bacterial and phage cRNA controls (1.5 pM BioB, 5 pM BioC, 25 pM BioD, and 100 pM Cre) to serve as internal controls for hybridization efficiency. Aliquots (200 μl) of the mixture were hybridized to arrays for 18 h at 45° C. in a GeneChip Hybridization Oven 640 (Affymetrix). Each array was washed and stained with streptavidin-phycoerythrin (Invitrogen) and amplified with biotinylated anti-streptavidin antibody (Vector Laboratories) on the GeneChip Fluidics Station 450 (Affymetrix). Arrays are scanned with the GeneArray G7 scanner (Affymetrix) to obtain image and signal intensities. The results suggest that six independent samples is sufficient to provide statistical power.

miRNA expression profiling—RNA labeling and hybridization on microarray chips were performed with miRNA isolated from wild type and LS/HNPCC mice. These studies were performed to reduce the miRNA array analysis from tissues to practice and to provide a statistical baseline for expression changes as well as sample power . Briefly, 5 μ g of total RNA from each sample was biotin-labeled by reverse transcription using 5′ biotin end-labeled random octomer oligo primer. Hybridization of biotin-labeled cDNA was carried out on a miRNA microarray chip (Ohio State University, Ver. 2.0), which contains 800 miRNA probes, including 245 human and 200 mouse miRNA genes, in quadruplicate. Hybridization signals were detected by a streptavidin-Alexa647 conjugate β using Axon Scanner 4000B and quantified by GENEPIX 6.0 software (Axon Instruments). The results show that six individual age-matched mouse tissue samples are sufficient for statistical power.

Validation of Microarray Results—The results of microarray analysis during experimental measurements may also be by alternative methods. The most frequently used method is real-time PCR. Validation of microarray results may be done for two reasons: first, to verify the observed changes and to ensure that they are reproducible in a larger number of samples; and second, to ensure that the array results did not result from inherent problems in the array technology or methods. Additionally, in certain embodiments, the relatively limited dynamic range of fluorescence detection in microarrays places limits on both sensitivity and specificity. The validation of expression profiling data is performed using either the Applied Biosystems 7900HT sequence detection system or the nCounter digital analysis system (NanoString Technologies) using pre-designed primers and reagents. Quantitative, computer-aided analysis is performed.

Antibody reagents exist to all of the protein products of the biomarker mRNAs. Moreover, 12 of these antibody reagents have been validated for IHC analysis. An alternate method to validate antibody reagents for use in western or IHC is constructing peptide-derived polyclonal antibodies to proteins biomarkers. This may be accomplished using the Ohio State University Comprehensive Cancer Center Core system. These antibody reagents are to examine the protein products of the mRNA whose expression pattern is altered in response to efficacious NSAIDs. Intestinal tissues for wild type and LS/HNPCC mice are examined for comparison. Addition pathway components identified that are related to the mRNA/protein biomarkers, are examined as to their expression, and post-translation modifications using appropriate antisera, by western and analysis. Relationships between mRNA expression alterations and the protein expression coded by those mRNAs are assessed.

Development of an LSIHNPCC Mouse Model

A mouse model where exon 12 of Msh2 is flanked by LoxP recombination sites (Msh2^{flox/flox}) was generated. Msh2 deletion was targeted to the intestinal epithelia by tissue-specific expression of Cre recombinase under the control of the villin promoter (VpC^{+/+}), which results in absent Msh2 protein in affected tissues. Tumorigenesis is confined to the intestines and has all of the pathological hallmarks of HNPCC-like tumors including MSI. When considering the utility of this mouse model it is important to note that while modern day LS/HNPCC largely occurs in the colon, it was originally described as a gastric cancer. Moreover, like their human counterparts, the LS/HNPCC mice develop (and ultimately succumb to) 1-2 tumors that appear to progress rapidly. This latter phenotype is one of the disadvantages of this LS/HNPCC mouse model since the effect of NSAIDs on tumor multiplicity is virtually impossible to determine. However, because the tumor numbers are small, even modest effects on tumor initiation and/or progression can have a demonstrated effect on the survival of these LS/HNPCC mice.

An alternative mouse model includes an azoxymethane-induced LS/HNPCC colon cancer model. This model used heterozygous Msh2^{lox/+} VpC^{+/+} mice that are phenotypically similar to human carriers.

The endpoint for the chemopreventive analysis of NSAIDs on mouse tumorigenesis is survival. Assessment of tumor kinetics, treatment regimes and pathological changes during tumor development constitute a correlative quantitative database.

Statistical Analysis

Bioinformatic and statistical analysis is performed in collaboration with the Center for Biostatistics at the Ohio State University (CBOSU). Survival is compared between chemopreventive drugs at each dose and an untreated control group. With n=20 mice per group per dose for each drug, there is calculated 80% power to detect a 30% survival difference at 1 year after treatment initiation. These calculations assume the approximate survival rate for each of the drug group is 50%, but for control group is 20%. Log-rank test is used for the survival analysis, and Kaplan-Meier curve is used to display the result. If significantly increased survival is observed for certain drug treatment, then the survival of those groups is compared as secondary. Other measurements, such as number of tumors, tumor locations, and pathology of tumors are considered as secondary endpoints. Number of tumors is compared using two-sample t-test, and descriptive statistics is provided for the other two measurements.

Microarray is used to investigate common mRNA and miRNA expression difference between tissues showing efficacious chemoprevention and tissues showing refractive chemoprevention with the same drug treatment. The OSU customized miRNA microarray chip is used for miRNA expression studies. The Affymetrix GeneChip is used for gene expression studies. A filtering method based on the requirement of a high percentage of arrays above a noise cutoff is applied to filter out low expression mRNA and miRNAs. A quantile normalization method is used for normalization, across arrays. Linear models are performed to detect differentially expressed genes. Gene expression level can be summarized over probe sets using the RMA method for mRNA expression. To improve the estimates of variability and statistical tests for differential expression, Variance shrinkage methods can be employed. The significance level is adjusted by controlling the mean number of false positives. To identify gene classes or pathways, EASE (National Institute of Allergy and Infectious Diseases, Bethesda, Md.), BRB array tools, and Ingenuity software (Ingenuity System, Redwood City, Calif.) are used. After filtering, approximately 10,000 mRNA or 700 miRNAs are testable on the chips. In this methodology, ten or five false positives for all testable mRNA or miRNAs are permitted. With n=6 mice per group per dose of each drug, there is more than 80% power to detect a 2-fold difference in mRNA and miRNA expression at a=0.001, assuming a CV=20% . Once the list of gene expression changes is identified for each drug at each dose compared to untreated, the common genes are identified. Correlation between the expression of interesting identified mRNAs and miRNAs and the dose effect on the expression of those genes, is investigated with linear mixed model as exploratory.

Further Methods and Definitions

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The terms “miR,” “mir” and “miRNA” generally refer to microRNA, a class of small RNA molecules that are capable of modulating RNA translation (see, Zeng and Cullen, RNA, 9(1):112-123, 2003; Kidner and Martienssen Trends Genet, 19(1):13-6, 2003; Dennis C, Nature, 420(6917):732, 2002; Couzin J, Science 298(5602):2296-7, 2002, each of which is incorporated by reference herein).

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary miRNA probes of the invention can be or be at least 60, 65, 70, 75, 80, 85, 90, 95, or 100% complementary to their target.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Control: A “control” refers to a sample or standard used for comparison with an experimental sample, such as a tumor sample obtained from a patient.

Decrease in survival: As used herein, “decrease in survival” refers to a decrease in the length of time before death of a patient, or an increase in the risk of death for the patient.

Detecting level of expression: For example, “detecting the level of miR or miRNA expression” refers to quantifying the amount of miR or miRNA present in a sample. Detecting expression of the specific miR, or any microRNA, can be achieved using any method known in the art or described herein, such as by qRT-PCR. Detecting expression of miR includes detecting expression of either a mature form of miRNA or a precursor form that is correlated with miRNA expression. Typically, miRNA detection methods involve sequence specific detection, such as by RT-PCR. miR-specific primers and probes can be designed using the precursor and mature miR nucleic acid sequences, which are known in the art.

MicroRNA (miRNA): Single-stranded RNA molecules that regulate gene expression. MicroRNAs are generally 21-23 nucleotides in length. MicroRNAs are processed from primary transcripts known as pri-miRNA to short stem-loop structures called precursor (pre)-miRNA and finally to functional, mature microRNA. Mature microRNA molecules arc partially-complementary to one or more messenger RNA molecules, and their primary function is to down-regulate gene expression. MicroRNAs regulate gene expression through the RNAi pathway.

miR expression: As used herein, “low miR expression” and “high miR expression” are relative terms that refer to the level of miRNAs found in a sample. In some embodiments, low and high miR expression is determined by comparison of miRNA levels in a group of control samples and test samples. Low and high expression can then be assigned to each sample based on whether the expression of mi in a sample is above (high) or below (low) the average or median miR expression level. For individual samples, high or low miR expression can be determined by comparison of the sample to a control or reference sample known to have high or low expression, or by comparison to a standard value. Low and high miR expression can include expression of either the precursor or mature forms of miRNA, or both.

Subject: As used herein, the term “subject” includes human and non-human animals. The preferred patient for treatment is a human. “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Screening: As used herein, “screening” refers to the process used to evaluate and identify candidate agents that affect such disease. Expression of a microRNA can be quantified using any one of a number of techniques known in the art and described herein, such as by microarray analysis or by qRT-PCR.

Small molecule: A molecule, typically with a molecular weight less than about 1000 Daltons, or in some embodiments, less than about 500 Daltons, wherein the molecule is capable of modulating, to some measurable extent, an activity of a target molecule.

Therapeutic: A generic term that includes both diagnosis and treatment.

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject.

As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically-effective amount: A quantity of a specified pharmaceutical or therapeutic agent sufficient to achieve a desired effect in a subject, or in a cell, being treated with the agent. The effective amount of the agent will be dependent on several factors, including, but not limited to the subject or cells being treated, and the manner of administration of the therapeutic composition.

In some embodiments of the present methods, use of a control is desirable. In that regard, the control may be a non-cancerous cell/tissue sample obtained from the same patient, or a cell/tissue sample obtained from a healthy subject, such as a healthy tissue donor. In another example, the control is a standard calculated from historical values. Tumor samples and non-cancerous cell/tissue samples can be obtained according to any method known in the art. For example, tumor and non-cancerous samples can be obtained from cancer patients that have undergone resection, or they can be obtained by extraction using a hypodermic needle, by microdissection, or by laser capture. Control (non-cancerous) samples can be obtained, for example, from a cadaveric donor or from a healthy donor.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having to be processed from the miR precursor. When a microRNA is referred to herein by name, the name corresponds to both the precursor and mature forms, unless otherwise indicated.

The level of at least one miR gene product can be measured in cells of a biological sample obtained from the subject. For example, a tissue sample can be removed from a subject suspected of having ovarian cancer, by conventional biopsy techniques. In another embodiment, a blood sample can be removed from the subject, and white blood cells can be isolated for DNA extraction by standard techniques. The blood or tissue sample is preferably obtained from the subject prior to initiation of radiotherapy, chemotherapy or other therapeutic treatment. A corresponding control tissue or blood sample, or a control reference sample, can be obtained from unaffected tissues of the subject, from a normal human individual or population of normal individuals, or from cultured cells corresponding to the majority of cells in the subject's sample. The control tissue or blood sample is then processed along with the sample from the subject, so that the levels of miR gene product produced from a given miR gene in cells from the subject's sample can be compared to the corresponding miR gene product levels from cells of the control sample. Alternatively, a reference sample can be obtained and processed separately (e.g., at a different time) from the test sample and the level of a miR gene product produced from a given miR gene in cells from the test sample can be compared to the corresponding miR gene product level from the reference sample.

In one embodiment, the level of the at least one miR gene product in the test sample is greater than the level of the corresponding miR gene product in the control sample (i.e., expression of the miR gene product is “up-regulated” or “increased”). As used herein, expression of a miR gene product is increased when the amount of miR gene product in a cell or tissue sample from a subject is greater than the amount of the same gene product in a control cell or tissue sample. In another embodiment, the level of the at least one miR gene product in the test sample is less than the level of the corresponding miR gene product in the control sample (i.e., expression of the miR gene product is “down-regulated” or “decreased”). As used herein, expression of a miR gene is decreased when the amount of miR gene product produced from that gene in a cell or tissue sample from a subject is less than the amount produced from the same gene in a control cell or tissue sample. The relative miR gene expression in the control and normal samples can be determined with respect to one or more RNA expression standards. The standards can comprise, for example, a zero miR gene expression level, the miR gene expression level in a standard cell line, the miR gene expression level in unaffected tissues of the subject, or the average level of miR gene expression previously obtained for a population of normal human controls.

An alteration (i.e., an increase or decrease) in the level of a miR gene product in the sample obtained from the subject, relative to the level of a corresponding miR gene product in a control sample, is indicative of the presence of ovarian cancer in the subject. In one embodiment, the level of at least one miR gene product in the test sample is greater than the level of the corresponding miR gene product in the control sample. In another embodiment, the level of at least one miR gene product in the test sample is less than the level of the corresponding miR gene product in the control sample.

In a certain embodiment, the at least one miR gene product is selected from the groups as shown in the Tables and Figures herein.

The level of a miR gene product in a sample can be measured using any technique that is suitable for detecting RNA expression levels in a biological sample. Suitable techniques (e.g., Northern blot analysis, RT-PCR, in situ hybridization) for determining RNA expression levels in a biological sample (e.g., cells, tissues) are well known to those of skill in the art. In a particular embodiment, the level of at least one miR gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes (e.g., DNA probes, RNA probes) for Northern blot hybridization of a given miR gene product can be produced from the nucleic acid sequences provided in the Tables herein and include, but are not limited to, probes having at least about 70%, 75%, 80%, 85%, 90%, 95%, 98% or 99% complementarily to a miR gene product of interest, as well as probes that have complete complementarity to a miR gene product of interest. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as ³H, ³²P, ³³P, ¹⁴C, ³⁵S; a heavy metal; a ligand capable of functioning as a specific binding pair member for a labeled ligand (e.g., biotin, avidin or an antibody); a fluorescent molecule; a chemiluminescent molecule; an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are incorporated herein by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of miR gene transcript levels. Using another approach, miR gene transcript levels can be quantified by computerized imaging systems, such as the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given miR gene product can be produced from the nucleic acid sequences provided in the Tables herein, and include, but are not limited to, probes having at least about 70%, 75%, 80%, 85%, 90%, 95%, 98% or 99% complementarity to a miR gene product of interest, as well as probes that have complete complementarity to a miR gene product of interest, as described above.

The relative number of miR gene transcripts in cells can also be determined by reverse transcription of miR gene transcripts, followed by amplification of the reverse-transcribed transcripts by polymerase chain reaction (RT-PCR). The levels of miR gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). Methods for performing quantitative and semi-quantitative RT-PCR, and variations thereof, are well known to those of skill in the art.

In some instances, it may be desirable to simultaneously determine the expression level of a plurality of different miR gene products in a sample. In other instances, it may be desirable to determine the expression level of the transcripts of all known miR genes correlated with a cancer. Assessing cancer-specific expression levels for hundreds of miR genes or gene products is time consuming and requires a large amount of total RNA (e.g., at least 20 μg for each Northern blot) and autoradiographic techniques that require radioactive isotopes.

To overcome these limitations, an oligolibrary, in microchip format (i.e., a microarray), may be constructed containing a set of oligonucleotide (e.g., oligodeoxynucleotide) probes that are specific for a set of miR genes. Using such a microarray, the expression level of multiple microRNAs in a biological sample can be determined by reverse transcribing the RNAs to generate a set of target oligodeoxynucleotides, and hybridizing them to probe the oligonucleotides on the microarray to generate a hybridization, or expression, profile. The hybridization profile of the test sample can then be compared to that of a control sample to determine which microRNAs have an altered expression level in ovarian cancer cells. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “miR-specific probe oligonucleotide” or “probe oligonucleotide specific for a miR” is meant a probe oligonucleotide that has a sequence selected to hybridize to a specific miR gene product, or to a reverse transcript of the specific miR gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal tissue may be distinguished from cancer cells, and within cancer cell types, different prognosis states (for example, good or poor long term survival prospects) may be determined. By comparing expression profiles of ovarian cells in different states, information regarding which genes are important (including both up- and down-regulation of genes) in each of these states is obtained. The identification of sequences that are differentially expressed in cancer cells or normal cells, as well as differential expression resulting in different prognostic outcomes, allows the use of this information in a number of ways. For example, a particular treatment regime may be evaluated (e.g., to determine whether a chemotherapeutic drug acts to improve the long-term prognosis in a particular patient). Similarly, diagnosis may be done or confirmed by comparing patient samples with known expression profiles. Furthermore, these gene expression profiles (or individual genes) allow screening of drug candidates that suppress the miR or disease expression profile or convert a poor prognosis profile to a better prognosis profile.

The microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs and other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6X SSPE/30% formamide at 25° C. for 18 hours, followed by washing in 0.75X TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool would allow for analysis of trans-species expression for each known miR under various conditions.

In addition to use for quantitative expression level assays of specific miRs, a microchip containing miRNA-specific probe oligonucleotides corresponding to a substantial portion of the miRNome, preferably the entire miRNome, may be employed to carry out miR gene expression profiling, for analysis of miR expression patterns. Distinct miR signatures can be associated with established disease markers, or directly with a disease state.

According to the expression profiling methods described herein, total RNA from a sample from a subject suspected of having a cancer is quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal, e.g., noncancerous, control sample. An alteration in the signal is indicative of the presence of, or propensity to develop, cancer in the subject. Alternatively, the sample profile may be compared to the hybridization profile generated from a cancer of known type or etiology.

In certain embodiments, the level of the at least one miR gene product is measured by reverse transcribing RNA from a test sample obtained from the subject to provide a set of target oligodeoxynucleotides, hybridizing the target oligodeoxynucleotides to a microarray that comprises miRNA-specific probe oligonucleotides to provide a hybridization profile for the test sample, and comparing the test sample hybridization profile to a hybridization profile generated from a control sample.

Other techniques for measuring miR gene expression are also within the skill in the art, and include various techniques for measuring rates of RNA transcription and degradation.

As defined herein, a “variant” of a miR gene product refers to a miRNA that has less than 100% identity to a corresponding wild-type miR gene product and possesses one or more biological activities of the corresponding wild-type miR gene product. Examples of such biological activities include, but are not limited to, inhibition of expression of a target RNA molecule (e.g., inhibiting translation of a target RNA molecule, modulating the stability of a target RNA molecule, inhibiting processing of a target RNA molecule) and inhibition of a cellular process associated with ovarian (e.g., cell differentiation, cell growth, cell death). These variants include species variants and variants that are the consequence of one or more mutations (e.g., a substitution, a deletion, an insertion) in a miR gene. In certain embodiments, the variant is at least about 70%, 75%, 80%, 85%, 90%, 95%, 98%, or 99% identical to a corresponding wild-type miR gene product.

As defined herein, a “biologically-active fragment” of a miR gene product refers to an RNA fragment of a miR gene product that possesses one or more biological activities of a corresponding wild-type miR gene product. As described above, examples of such biological activities include, but are not limited to, inhibition of expression of a target RNA molecule and inhibition of a cellular process associated with cancer. In certain embodiments, the biologically-active fragment is at least about 5, 7, 10, 12, 15, or 17 nucleotides in length. In a particular embodiment, an isolated miR gene product can be administered to a subject in combination with one or more additional anti-cancer treatments. Suitable anti-cancer treatments include, but are not limited to, chemotherapy, hormonal therapy, radiation therapy and combinations (e.g., chemoradiation).

Embodiments of the invention may be used to select appropriate drugs and tailor the treatment of patients based on predictions and indications of the responsiveness of the tumor or cellular environment to selected treatments.

The terms “treat”, “treating” and “treatment”, as used herein, refer to ameliorating symptoms associated with a disease or condition, for example, ovarian cancer, including preventing or delaying the onset of the disease symptoms, and/or lessening the severity or frequency of symptoms of the disease or condition. The terms “subject” and “individual” are defined herein to include animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “effective amount” of an isolated miR gene product is an amount sufficient to inhibit proliferation of a cancer cell in a subject suffering from ovarian cancer. One skilled in the art can readily determine an effective amount of a miR gene product to be administered to a given subject, by taking into account factors, such as the size and weight of the subject; the extent of disease penetration; the age, health and sex of the subject; the route of administration; and whether the administration is regional or systemic.

For example, an effective amount of an isolated miR gene product can be based on the approximate weight of a tumor mass to be treated. The approximate weight of a tumor mass can be determined by calculating the approximate volume of the mass, wherein one cubic centimeter of volume is roughly equivalent to one gram. An effective amount of the isolated miR gene product based on the weight of a tumor mass can be in the range of about 10-500 micrograms/gram of tumor mass. In certain embodiments, the tumor mass can be at least about 10 micrograms/gram of tumor mass, at least about 60 micrograms/gram of tumor mass or at least about 100 micrograms/gram of tumor mass.

An effective amount of an isolated miR gene product can also be based on the approximate or estimated body weight of a subject to be treated. Preferably, such effective amounts are administered parenterally or enterally, as described herein. For example, an effective amount of the isolated miR gene product that is administered to a subject can range from about 5-3000 micrograms/kg of body weight, from about 700-1000 micrograms/kg of body weight, or greater than about 1000 micrograms/kg of body weight.

One skilled in the art can also readily determine an appropriate dosage regimen for the administration of an isolated miR gene product to a given subject. For example, a miR gene product can be administered to the subject once (e.g., as a single injection or deposition). Alternatively, a miR gene product can be administered once or twice daily to a subject for a period of from about three to about twenty-eight days, more particularly from about seven to about ten days. In a particular dosage regimen, a miR gene product is administered once a day for seven days. Where a dosage regimen comprises multiple administrations, it is understood that the effective amount of the miR gene product administered to the subject can comprise the total amount of gene product administered over the entire dosage regimen.

As used herein, an “isolated” miR gene product is one that is synthesized, or altered or removed from the natural state through human intervention. For example, a synthetic miR gene product, or a miR gene product partially or completely separated from the coexisting materials of its natural state, is considered to be “isolated.” An isolated miR gene product can exist in a substantially-purified form, or can exist in a cell into which the miR gene product has been delivered. Thus, a miR gene product that is deliberately delivered to, or expressed in, a cell is considered an “isolated” miR gene product. A miR gene product produced inside a cell from a miR precursor molecule is also considered to be an “isolated” molecule. According to the invention, the isolated miR gene products described herein can be used for the manufacture of a medicament for treating a cancer in a subject (e.g., a human or animal).

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGencs (Ashland, Mass., U.S.A.) and Cruachem (Glasgow, UK).

Alternatively, the miR gene products can be expressed from recombinant circular or linear DNA plasmids using any suitable promoter. Suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the miR gene products in cancer cells.

The miR gene products that are expressed from recombinant plasmids can be isolated from cultured cell expression systems by standard techniques. The miR gene products that are expressed from recombinant plasmids can also be delivered to, and expressed directly in, the cancer cells. The use of recombinant plasmids to deliver the miR gene products to cancer cells is discussed in more detail below.

The miR gene products can be expressed from a separate recombinant plasmid, or they can be expressed from the same recombinant plasmid. In one embodiment, the miR gene products are expressed as RNA precursor molecules from a single plasmid, and the precursor molecules are processed into the functional miR gene product by a suitable processing system, including, but not limited to, processing systems extant within a cancer cell. Other suitable processing systems include, e.g., the in vitro Drosophila cell lysate system (e.g., as described in U.S. Published Patent Application No. 2002/0086356 to Tuschl et al., the entire disclosure of which is incorporated herein by reference) and the E. coli RNAse III system (e.g., as described in U.S. Published Patent Applicaton No. 2004/0014113 to Yang et al., the entire disclosure of which is incorporated herein by reference).

Selection of plasmids suitable for expressing the miR gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are incorporated herein by reference.

In one embodiment, a plasmid expressing the miR gene products comprises a sequence encoding a miR precursor RNA under the control of the CMV intermediate-early promoter. As used herein, “under the control” of a promoter means that the nucleic acid sequences encoding the miR gene product are located 3′ of the promoter, so that the promoter can initiate transcription of the miR gene product coding sequences.

The miR gene products can also be expressed from recombinant viral vectors. It is contemplated that the miR gene products can be expressed from two separate recombinant viral vectors, or from the same viral vector. The RNA expressed from the recombinant viral vectors can either be isolated from cultured cell expression systems by standard techniques, or can be expressed directly in cancer cells. The use of recombinant viral vectors to deliver the miR gene products to cancer cells is discussed in more detail below.

The recombinant viral vectors of the invention comprise sequences encoding the miR gene products and any suitable promoter for expressing the RNA sequences. Suitable promoters include, but are not limited to, the U6 or H1 RNA pol III promoter sequences, or the cytomegalovirus promoters. Selection of other suitable promoters is within the skill in the art. The recombinant viral vectors of the invention can also comprise inducible or regulatable promoters for expression of the miR gene products in a cancer cell.

Any viral vector capable of accepting the coding sequences for the miR gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors that express different capsid protein serotypes are within the skill in the art; see, e.g., Rabinowitz, J. E., et al. (2002), J. Virol. 76:791-801, the entire disclosure of which is incorporated herein by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are incorporated herein by reference.

Particularly suitable viral vectors are those derived from AV and AAV. A suitable AV vector for expressing the miR gene, products, a method for constructing the recombinant AV vector, and a method for delivering the vector into target cells, are described in Xia et al. (2002), Nat. Biotech. 20:1006-1010, the entire disclosure of which is incorporated herein by reference. Suitable AAV vectors for expressing the miR gene products, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are incorporated herein by reference. In one embodiment, the miR gene products are expressed from a single recombinant AAV vector comprising the CMV intermediate early promoter.

In a certain embodiment, a recombinant AAV viral vector of the invention comprises a nucleic acid sequence encoding a miR precursor RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the miR sequences from the vector, the polyT termination signals act to terminate transcription.

In other embodiments of the treatment methods of the invention, an effective amount of at least one compound that inhibits miR expression can be administered to the subject. As used herein, “inhibiting miR expression” means that the production of the precursor and/or active, mature form of miR gene product after treatment is less than the amount produced prior to treatment. One skilled in the art can readily determine whether miR expression has been inhibited in a cancer cell, using, for example, the techniques for determining miR transcript level discussed herein. Inhibition can occur at the level of gene expression (i.e., by inhibiting transcription of a miR gene encoding the miR gene product) or at the level of processing (e.g., by inhibiting processing of a miR precursor into a mature, active miR).

As used herein, an “effective amount” of a compound that inhibits miR expression is an amount sufficient to inhibit proliferation of a cancer cell in a subject suffering from a cancer (e.g., ovarian cancer). One skilled in the art can readily determine an effective amount of a miR expression-inhibiting compound to be administered to a given subject, by taking into account factors, such as the size and weight of the subject; the extent of disease penetration; the age, health and sex of the subject; the route of administration; and whether the administration is regional or systemic.

For example, an effective amount of the expression-inhibiting compound can be based on the approximate weight of a tumor mass to be treated, as described herein. An effective amount of a compound that inhibits miR expression can also be based on the approximate or estimated body weight of a subject to be treated, as described herein.

One skilled in the art can also readily determine an appropriate dosage regimen for administering a compound that inhibits miR expression to a given subject, as described herein.

Suitable compounds for inhibiting miR gene expression include double-stranded RNA (such as short- or small-interfering RNA or “siRNA”), antisense nucleic acids, and enzymatic RNA molecules, such as ribozymes. Each of these compounds can be targeted to a given miR gene product and interfere with the expression (e.g., by inhibiting translation, by inducing cleavage and/or degradation) of the target miR gene product.

For example, expression of a given miR gene can be inhibited by inducing RNA interference of the miR gene with an isolated double-stranded RNA (“dsRNA”) molecule which has at least 90%, for example at least 95%, at least 98%, at least 99%, or 100%, sequence homology with at least a portion of the miR gene product. In a particular embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence that is substantially identical to a nucleic acid sequence contained within the target miR gene product.

As used herein, a nucleic acid sequence in an siRNA that is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3′ overhang” refers to at least one unpaired nucleotide extending from the 3′-end of a duplexed RNA strand. Thus, in certain embodiments, the siRNA comprises at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated miR gene products. Exemplary methods for producing and testing dsRNA or siRNA molecules are described in U.S. Published Patent Application. No. 2002/0173478 to Gewirtz and in U.S. Published Patent Application No. 2004/0018176 to Reich et al., the entire disclosures of both of which are incorporated herein by reference.

Expression of a given miR gene can also be inhibited by an antisense nucleic acid. As used herein, an “antisense nucleic acid” refers to a nucleic acid molecule that binds to target RNA by means of RNA-RNA, RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, peptide nucleic acids (PNA)) that generally comprise a nucleic acid sequence complementary to a contiguous nucleic acid sequence in a miR gene product. The antisense nucleic acid can comprise a nucleic acid sequence that is 50-100% complementary, 75-100% complementary, or 95-100% complementary to a contiguous nucleic acid sequence in a miR gene product. Nucleic acid sequences of particular human miR gene products are provided in the Tables herein. Without wishing to be bound by any theory, it is believed that the antisense nucleic acids activate RNase H or another cellular nuclease that digests the miR gene product/antisense nucleic acid duplex.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators, such as acridine, or one or more nuclease-resistant groups.

Antisense nucleic acids can be produced chemically or biologically, or can be expressed from a recombinant plasmid or viral vector, as described above for the isolated miR gene products. Exemplary methods for producing and testing are within the skill in the art; see, e.g., Stein and Cheng (1993), Science 261:1004 and U.S. Pat. No. 5,849,902 to Woolf et al., the entire disclosures of which are incorporated herein by reference.

Expression of a given miR gene can also be inhibited by an enzymatic nucleic acid. As used herein, an “enzymatic nucleic acid” refers to a nucleic acid comprising a substrate binding region that has complementarity to a contiguous nucleic acid sequence of a miR gene product, and which is able to specifically cleave the miR gene product. The enzymatic nucleic acid substrate binding region can be, for example, 50-100% complementary, 75-100% complementary, or 95-100% complementary to a contiguous nucleic acid sequence in a miR gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated miR gene products. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are incorporated herein by reference.

Administration of at least one miR gene product, or at least one compound for inhibiting miR expression, will inhibit the proliferation of cancer cells in a subject who has a cancer. As used herein, to “inhibit the proliferation of a cancer cell” means to kill the cell, or permanently or temporarily arrest or slow the growth or reproduction of the cell. Inhibition of cancer cell proliferation can be inferred if the number of such cells in the subject remains constant or decreases after administration of the miR gene products or miR gene expression-inhibiting compounds. An inhibition of cancer cell proliferation can also be inferred if the absolute number of such cells increases, but the rate of tumor growth decreases.

The number of cancer cells in the body of a subject can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

The miR gene products or miR gene expression-inhibiting compounds can be administered to a subject by any means suitable for delivering these compounds to cancer cells of the subject. For example, the miR gene products or miR expression-inhibiting compounds can be administered by methods suitable to transfect cells of the subject with these compounds, or with nucleic acids comprising sequences encoding these compounds. In one embodiment, the cells are transfected with a plasmid or viral vector comprising sequences encoding at least one miR gene product or miR gene expression-inhibiting compound.

Transfection methods for eukaryotic cells are well known in the art, and include, e.g., direct injection of the nucleic acid into the nucleus or pronucleus of a cell; electroporation; liposome transfer or transfer mediated by lipophilic materials; receptor-mediated nucleic acid delivery, bioballistic or particle acceleration; calcium phosphate precipitation, and transfection mediated by viral vectors.

For example, cells can be transfected with a liposomal transfer compound, e.g., DOTAP (N-[1-(2,3-dioleoyloxy)propyl]-N,N,N-trimethyl-ammonium methylsulfate, Boehringer-Mannheim) or an equivalent, such as LIPOFECTIN. The amount of nucleic acid used is not critical to the practice of the invention; acceptable results may be achieved with 0.1-100 micrograms of nucleic acid/10⁵ cells. For example, a ratio of about 0.5 micrograms of plasmid vector in 3 micrograms of DOTAP per 10⁵ cells can be used.

A miR gene product or miR gene expression-inhibiting compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and direct injection into the tumor.

In the present methods, a miR gene product or miR gene product expression-inhibiting compound can be administered to the subject either as naked RNA, in combination with a delivery reagent, or as a nucleic acid (e.g., a recombinant plasmid or viral vector) comprising sequences that express the miR gene product or miR gene expression-inhibiting compound. Suitable delivery reagents include, e.g., the Mirus Transit TKO lipophilic reagent; LIPOFECTIN; lipofectamine; cellfectin; polycations (e.g., polylysine) and liposomes.

Recombinant plasmids and viral vectors comprising sequences that express the miR gene products or miR gene expression-inhibiting compounds, and techniques for delivering such plasmids and vectors to cancer cells, are discussed herein and/or are well known in the art.

In a particular embodiment, liposomes are used to deliver a miR gene product or miR gene expression-inhibiting compound (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of factors, such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are incorporated herein by reference.

The liposomes for use in the present methods can comprise a ligand molecule that targets the liposome to cancer cells. Ligands that bind to receptors prevalent in cancer cells, such as monoclonal antibodies that bind to tumor cell antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the Liposome structure. In a particularly preferred embodiment, a liposome of the invention can comprise both an opsonization-inhibition moiety and a ligand.

Opsonization-inhibiting moieties for use in preparing the liposomes of the invention are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is incorporated herein by reference.

Opsonization-inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) or derivatives thereof; e.g., methoxy PEG or PPG, and PEG or PPG stearate; synthetic polymers, such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization-inhibiting polymer can be a block copolymer of PEG and either a polyamino acid, polysaccharide, polyamidoamine, polyethyleneamine, or polynucleotide. The opsonization-inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or a derivative thereof. Liposomes modified with PEG or PEG-derivatives are sometimes called “PEGylated liposomes.”

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example, solid tumors (e.g., ovarian cancers), will efficiently accumulate these liposomes; see Gabizon, et al. (1988), Proc. Natl. Acad. Sci., U.S.A., 18:6949-53. In addition, the reduced uptake by the RES lowers the toxicity of stealth Liposomes by preventing significant accumulation of the liposomes in the liver and spleen. Thus, liposomes that are modified with opsonization-inhibition moieties are particularly suited to deliver the miR gene products or miR gene expression-inhibition compounds (or nucleic acids comprising sequences encoding them) to tumor cells.

The miR gene products or miR gene expression-inhibition compounds can be formulated as pharmaceutical compositions, sometimes called “medicaments,” prior to administering them to a subject, according to techniques known in the art. Accordingly, the invention encompasses pharmaceutical compositions for treating cancer. In one embodiment, the pharmaceutical composition comprises at least one isolated miR gene product, or an isolated variant or biologically-active fragment thereof, and a pharmaceutically-acceptable carrier. In a particular embodiment, the at least one miR gene product corresponds to a miR gene product that has a decreased level of expression in cancer cells relative to suitable control cells.

Kits

Any of the compositions described herein may be comprised in a kit. In a non-limiting example, reagents for isolating miRNA, labeling miRNA, and/or evaluating a miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, and components for isolating miRNA. Other kits may include components for making a nucleic acid array comprising oligonucleotides complementary to miRNAs, and thus, may include, for example, a solid support.

For any kit embodiment, including an array, there can be nucleic acid molecules that contain a sequence that is identical or complementary to all or part of any of the miRs described herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miRNA from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled miRNA. The kit may also include components that preserve or maintain the miRNA or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of miRNA.

It is also contemplated that any embodiment discussed in the context of a miRNA array may be employed more generally in screening or profiling methods or kits of the invention. In other words, any embodiments describing what may be included in a particular array can be practiced in the context of miRNA profiling more generally and need not involve an array per se.

It is also contemplated that any kit, array or other detection technique or tool, or any method can involve profiling for any of these miRNAs. Also, it is contemplated that any embodiment discussed in the context of an miRNA array can be implemented with or without the array format in methods of the invention; in other words, any miRNA in an miRNA array may be screened or evaluated in any method of the invention according to any techniques known to those of skill in the art. The array format is not required for the screening and diagnostic methods to be implemented.

The kits for using miRNA arrays for therapeutic, prognostic, or diagnostic applications and such uses are contemplated. The kits can include a miRNA array, as well as information regarding a standard or normalized miRNA profile for the miRNAs on the array. Also, in certain embodiments, control RNA or DNA can be included in the kit. The control RNA can be miRNA that can be used as a positive control for labeling and/or array analysis. Also, the sample can be blood or tissue.

In one embodiment, the kit for the characterization of cancer includes at least one detection probe for a miRNA or mRNA listed in FIG. 7. In certain embodiments, the kit is in the form of, or comprises, an oligonucleotide array.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Array Preparation and Screening

Also provided herein are the preparation and use of miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect miRNA; consequently, methods and compositions may be used with a variety of different types of miRNA arrays. In certain embodiments, the miR gene product comprises one or more of the miRs described herein.

Microarrays

The microarray can comprise oligonucleotide probes obtained from known or predicted miRNA sequences. The array may contain different oligonucleotide probes for each miRNA, for example one containing the active mature sequence and another being specific for the precursor of the miRNA. The array may also contain controls such as one or more sequences differing from the human orthologs by only a few bases, which can serve as controls for hybridization stringency conditions. It is also possible to include viral miRNAs or putative miRNAs as predicted from bioinformatic tools. Further, it is possible to include appropriate controls for non-specific hybridization on the microarray.

In certain embodiments, in a method for in vitro diagnosis of cancer chemopreventive therapy response or prognosis of progression, one or more miRNAs as ovarian cancer biomarkers are obtained from a method for selecting a miRNA or mRNA for use as a disease diagnostic biomarker, comprising: a) obtaining samples from subjects, wherein the subjects are composed of people suffering from the disease and people not suffering from the disease; b) determining expression levels of candidate biomarker miRNAs and/or mRNAs and an internal control RNA in the samples; c) computing relative expression levels of the biomarker(s); d) computing a prediction model with one or more variables, wherein the variables include relative expression levels of one or more biomarkers and, optionally one or more risk factors of the disease, including genetic factors; and e) computing a disease risk probability, sensitivity and specificity by the prediction model, wherein the one or more biomarkers with the highest sensitivity and the highest specificity are selected to be the disease diagnosis biomarker.

Citation of any documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1-10. (canceled)

11. A method of assessing the efficacy of NSAID cancer chemoprevention treatment in a human or animal subject, the method comprising:
obtaining a nucleic acid-containing sample from a subject receiving NSAID treatment;
measuring at least one mRNA and/or miR biomarker; and
determining whether the NSAID treatment is efficacious based on the expression of the at least one mRNA and/or miR biomarker.

12. The method claim 11 wherein the sample is extracted from at least one of the group consisting of: tissue, blood, tumor, stool, mucus, dysplastic tissue, colorectal tissue, ovarian tissue, urothelial tissue, endometrial tissue, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof.

13. The method of claim 11 wherein the measuring is performed by one or more methods selected from the group consisting of: hybridization assay, microarray chip, n-counter (NanoString) analysis, miR-Seq analysis, PCR, real-time PCR, microfluidic cards, oligonucleotide probes, and northern blot.

14. The method of claim 11 wherein the determining further comprises comparing the sample expression to a control level, wherein the control level is determined from measurements of control expression levels in tissue selected from the group consisting of: cancerous tissue, healthy tissue, and tissue taken from the subject at an earlier time, wherein the earlier time is greater than any of: one week, one month, six months, 200 days, one year, 400 days, eighteen months, 600 days, two years, three years, four years, five years, six years, seven years, eight years, nine years, ten years, or eleven years.

15. A method of inhibiting tumors that display microsatellite instability (MSI) in a human or animal subject comprising administering an efficacious NSAID for a period exceeding five years.

16. The method of claim 15 wherein the NSAID is Naproxen.

17. A method of inhibiting tumor incidence, or inhibiting tumor progression, in hereditary non-polyposis colorectal cancer or Lynch syndrome (LS/HNPCC) comprising:
administering a therapeutically effective dose of a non-steroidal anti-inflammatory drug (NSAID), wherein the NSAID is Naproxen and/or naproxen sodium.

18. A method of inhibiting tumor incidence, or inhibiting tumor progression, in LS/HNPCC comprising:
administering a therapeutically effective dose of a non-steroidal anti-inflammatory drug (NSAID); and
monitoring the efficacy of the treatment by measuring at least one biomarker selected from the group consisting of: an activation miR, an inhibition miR, an activation mRNA, and an inhibition mRNA.

19-20. (canceled)

21. A method of treating LS/HNPCC comprising:
administering a therapeutically effective dose of a non-steroidal anti-inflammatory drug (NSAID); and
monitoring the efficacy of the treatment by measuring at least one biomarker selected from the group consisting of: an activation miR, an inhibition miR, an activation mRNA, and an inhibition mRNA.

22. The method of claim 21, wherein the NSAID is naproxen and/or naproxen sodium.

23. The method of claim 21 wherein the NSAID is selected from the group consisting of: naproxen, naproxen sodium, nitric oxide-donating acetylsalicylic acid, sulindac, nitric oxide-donating sulindac, fenoprofen, ketoprofen, oxaprozin, indomethacin, etodolac, diclofenac, piroxicam, meloxicam, tenoxicam, droxicam, lomoxicam, isoxicam, mefenamic acid, meclofenamic acid, flufenamic acid, tolfenamic acid, celecoxicab, rofecoxib, valdecoxib, parecoxib, lumiracoxib, etoricoxib, nimesulide, and niflumic acid, and licofenac.

24. The method of any one of claim 21 wherein at least two NSAIDs are administered.

25. The method of claim 21, further comprising
increasing or decreasing the dose to therapeutically effective levels, whereby the therapeutically effective level is determined by comparing the at least one biomarker with a control.

26. A method for screening a NSAID compound comprising:
administering a test NSAID compound to an animal subject;
measuring at least one biomarker selected from the group consisting of: an activation miR, an inhibition miR, an activation mRNA, and an inhibition mRNA; and
comparing the test compound biomarker expression profile with a biomarker expression profile for a NSAID compound of tested efficacy.

27. A method for the assessment of a clinical condition related to cancer of a patient for in vitro diagnosis of cancer, comprising:
a) obtaining a sample from a subject;
b) determining expression levels of one or more miRNAs as cancer biomarkers and an internal control RNA;
c) computing relative expression levels of the one or more miRNAs as cancer biomarkers;
d) computing a prediction model by using one or more variables, wherein the variables include relative expression levels of the one or more miRNAs as cancer biomarkers; and,
e) computing a cancer progression risk probability by the prediction model, wherein the subject is diagnosed as at risk for cancer progression if the disease risk probability is greater than 0.5.

28. A method for providing a prognosis for survival in a subject having LS/HNPCC, or to determine progression of LS/HNPCC in a subject, the method comprising:
comparing the level of at least one biomarker in a sample to a control, wherein the at least one biomarker is chosen from: an activation miR, an inhibition miR, an activation mRNA, an inhibition mRNA, and combinations thereof; and
determining whether the level of the at least one biomarker is reduced or whether the level of the at least one biomarker is altered in the sample, as compared to the control, thereby characterizing a prognosis for survival in a subject having LS/HNPCC.

29. (canceled)

30. A diagnostic kit of molecular markers for providing a prognosis for poor survival of a subject having LS/HNPCC, according to the method of claim 28, the kit comprising a plurality of nucleic acid molecules, each nucleic acid molecule encoding a microRNA and/or messenger RNA sequence,
wherein at least one miRNA sequence is miR-103; and
wherein one or more of the plurality of nucleic acid molecules are differentially expressed in the target cells and in one or more control cells, and wherein the one or more differentially expressed nucleic acid molecules together represent a nucleic acid expression signature that is indicative of a prognosis of poor survival in a subject having LS/HNPCC.

31. A method for characterizing a disease in a subject having, or suspected of having, LS/HNPCC comprising:
comparing the level of at least one biomarker in a sample to a control, wherein the at least one biomarker is chosen from: an activation miR, an inhibition miR, an activation mRNA, an inhibition mRNA, and combinations thereof; and,
determining whether the level of the at least one biomarker is reduced or whether the level of the at least one biomarker is elevated in the sample, as compared to the control, thereby characterizing the disease in the subject.

32. The method of claim 11, wherein the at least one mRNA and/or miR biomarker is at least one activation miR selected from the group consisting of: miR-103, miR-193, miR-128, miR-136, miR-199a-3p, miR-27b, miR-377, miR-21, miR-34a, miR-328, and miR-296-3p; and the NSAID treatment is efficacious if the activation miR measured in the sample is activated.

33. The method of claim 32, wherein the at least one activation miR includes miR-103, mir-193, and mir-128.

34. The method of claim 11, wherein the at least one mRNA and/or miR biomarker is at least one inhibition miR selected from the group consisting of: miR-341, miR-297c, miR-423-5p, miR762; miR-2135, miR-423-5p, and miR-297c; and the NSAID treatment is efficacious if the inhibition miR measured in the sample is inhibited.

35. The method of claim 11, wherein the at least one mRNA and/or miR biomarker is at least one activation miR selected from the group consisting of: miR-19a, miR-126-5p, miR-29b, miR-301a, miR-1, miR-218, miR-142-3p, miR-190, miR-144, miR-33, miR-377, and miR-1196; and the NSAID treatment is efficacious if the activation miR measured in the sample is activated.

36. The method of claim 11, wherein the at least one mRNA and/or miR biomarker is at least one activation miR selected from the group consisting of: miR-375-A, miR-615-P, miR-706-P, miR-485-3p-A, miR-290-P, miR-543-A, miR-425-3p-A, miR-326-A, miR-589-A, miR-129-2-A, miR-553-A, miR-96-A, miR-7-2-A, miR-519e*-5p-A, miR-293-A, miR-551a-A, miR-594-P, miR-601-A, miR-487b-A, miR-676-3p-A, and miR-500-P; and the NSAID treatment is efficacious if the activation miR measured in the sample is activated.

37. The method of claim 11, wherein the at least one mRNA and/or miR biomarker is at least one activation miR selected from the group consisting of: miR-103, miR-193, miR-128, miR-136, miR-199a-3p, miR-27b, miR-377, miR-21, miR-34a, miR-328, miR-296-3p, miR-19a, miR-126-5p, miR-29b, miR-301a, miR-1, miR-218, miR-142-3p, miR-190, miR-144, miR-33, miR-377, miR-1196, miR-375-A, miR-615-P, miR-706-P, miR-485-3p-A, miR-290-P, miR-543-A, miR-425-3p-A, miR-326-A, miR-589-A, miR-129-2-A, miR-553-A, miR-96-A, miR-7-2-A, miR-519e*-5p-A, miR-293-A, miR-551-A, miR-594-P, miR-601-A, miR-487b-A, miR-676-3p-A, miR-500-P, miR-706, miR-543, miR-328, miR-202, miR-338, and miR-483; and the NSAID treatment is efficacious if the activation miR measured in the sample is activated.

38. The method of claim 11, wherein the at least one mRNA and/or miR biomarker is at least one activation mRNA selected from the group consisting of: RPL13A, Npas2, Arnt1, Kit1, Malat1, Ept1, 1810014B01Rik, Aes, Sepw1, Rab4a, bak1, mal1, Zfand5, Nfi13,Npas2, and Irf2bp21700110K17Rik; and the NSAID treatment is efficacious if the activation mRNA measured in the sample is activated.

39. The method of claim 38, wherein the at least one activation mRNA includes Kit1.

40. The method of claim 11, wherein the at least one mRNA and/or miR biomarker is at least one inhibition mRNA selected from the group consisting of: Slc38a10, Aes, Rab5c, Cfl1, PPARD, Actb, Ptt1ip, Espn, Elovl1, Angptl 2, Hlf, Per2, Tef, Per3, Dbp; Ste20a1, and Gm129; and the NSAID treatment is efficacious if the inhibition mRNA measured in the sample is inhibited.

41. The method of claim 40, further comprising measuring at least one inhibition mRNA selected from the group consisting of: Ang4; Bambi, Cd24a, Cc16, Entpd5, Wee1, Ank3, Trp53, Sel11, Ube4b, Arid5b, Prkab2, Perl, Bace1, and Stox2.
